(19) United States
(12) Patent Application Publication
(10) Pub. No.: US 2026/0335261 A1
(43) Pub. Date: Oct. 29, 2026

(54) METHODS AND COMPOSITIONS FOR CHRONIC INFLAMMATION USING COLCHICINE

(71) Applicant: Murray and Poole Enterprises, LTD., Gibraltar (GI)

(72) Inventor: Michael Riel, Dubai (AE)

(73) Assignee: Murray and Poole Enterprises, LTD., Gibraltar (GI)

(21) Appl. No.: 19/650,398

(22) Filed: Apr. 17, 2026

Related U.S. Application Data
(60) Provisional application No. 63/790,766, filed on Apr. 18, 2025.

(51) Int. Cl. A61K 31/165; A61K 9/20; A61K 45/06; A61P 29/00; G01N 33/68

(52) U.S. Cl. CPC A61K 31/165; A61K 9/2009; A61K 9/2013; A61K 9/2018; A61K 9/2054; A61K 9/2059; A61K 45/06; A61P 29/00; G01N 33/6863; G01N 33/6869; G01N 33/6893; G01N 2333/4737; G01N 2333/525; G01N 2333/5412; G01N 2800/52; G01N 2800/7095

(57) ABSTRACT

Pharmaceutical compositions of colchicine for once-daily oral administration are provided for use in the treatment of chronic inflammation, including inflammation arising after COVID-19 vaccination and/or COVID-19 infection (Long COVID). The formulations comprise, in some embodiments, an immediate-release component and/or a sustained-release component, which is tailored to achieve a desired pharmacokinetic profile suitable for long-term anti-inflammatory therapy. The disclosed compositions are designed to reduce systemic levels of key inflammatory biomarkers, such as high-sensitivity C-reactive protein (hs-CRP), C-reactive protein (CRP), interleukin-1 (IL-1), interleukin-1β (IL-1β), interleukin-6 (IL-6), tumor necrosis factor-alpha (TNF-α), and myeloperoxidase (MPO), and to alleviate clinical manifestations of persistent inflammation, including vaccine-associated myocarditis, pericarditis, or other inflammatory sequelae. Methods of treating post-vaccination and/or post-infection inflammatory syndromes, improving quality of life, and reducing hospitalization risk in mammalian subjects are also provided.

### BACKGROUND OF THE INVENTION

Colchicine, chemical name (−)—N-[(7S,12aS)-1,2,3,10-tetramethoxy-9-oxo-5,6,7,9-tetrahydrobenzo[a]heptalen-7-yl]-acetamide, is an alkaloid found in extracts of Colchicum autumnale, Gloriosa superba, and other plants. It is a microtubule-disrupting agent used in the treatment of conditions that may be treated, relieved, or prevented with anti-inflammatory treatment.

Colchicine is well recognized as a valid therapy for treatment of acute flares of gouty arthritis, familial Mediterranean fever (FMF), and Behçet's disease. It has also been used to treat many inflammatory disorders that lead to fibrosis. In the recent past, colchicine has been proposed to be effective in therapy for cardiovascular diseases.

In particular, colchicine has been proposed as a first treatment option for recurrent pericarditis (class I indication) and optionally for acute pericarditis (class Ila indication) in the 2004 European guidelines on the management of pericardial diseases (Maisch et al., “Guidelines on the Diagnosis and Management of Pericardial Diseases,” Eur. Heart J., 2004, 25:916-928).

Imazio et al., Circulation, 112(13):2012-2016, 2005, showed that colchicine was effective for the treatment and the prevention of recurrent pericarditis in a prospective, randomized, open-label designed study of 120 patients with a first episode of acute pericarditis (idiopathic, viral, postpericardiotomy syndromes, and connective tissue diseases), who were randomly assigned to conventional treatment with aspirin or conventional treatment plus colchicine (1.0 to 2.0 mg for the first day and then 0.5 to 1.0 mg/day for 3 months). The primary end point was recurrence rate, which was significantly reduced from 32.3% down to 10.7% at 18 months in the colchicine group (p=0.004).

Further, the same group showed that after conventional treatment failure colchicine efficiently managed acute pericarditis. (See, Imazio at al., Arch. Intern. Med., 165(17):1987-91, 2005). In a prospective, randomized, open-label design, 84 consecutive patients with a first episode of recurrent pericarditis were randomly assigned to receive conventional treatment with aspirin alone or conventional treatment plus colchicine (1.0-2.0 mg colchicine the first day, and then 0.5-1.0 mg/d colchicine for 6 months). The primary end point was the recurrence rate, which was significantly decreased in the colchicine group (actuarial rates at 18 months were 24.0% vs 50.6% with conventional treatment).

It has also been shown that colchicine is effective for secondary prevention of recurrent pericarditis. (See, Imazio et al., Ann. Intern. Med., 155(7):409-14, 2011). Colchicine has also been proposed to reduce post-pericardiotomy reactions revealed as pericarditis. (See, Imazio et al., Am. Heart J., 162(3):527-532, 2011; and Meurin and Tabet, Arch. Cardiovasc. Dis., 104(8-9):425-427, 2011).

Colchicine for the treatment of post-pericardiotomy syndrome (PPS) was tested for the first time in a preliminary prospective, open-label, randomized trial of colchicine (1.5 mg/day) compared with placebo beginning on the third post-operative day in 163 patients who underwent cardiac surgery. (See, Finkelstein et al., Herz, 27(8):791-194, 2002). This study concluded that colchicine may be effective in preventing PPS in patients after cardiac surgery. It is interesting to note that 52 of the 163 patients were excluded from the study due to postoperative complications, noncompliance, or more importantly, due to the known toxic gastrointestinal side effects of colchicine at this dosage level.

The effectiveness of colchicine for the prevention of PPS has also been shown in a multicentre, double-blind, randomized trial, in which 360 patients (mean age 65.7±12.3 years, 66% males), 180 in each treatment arm, were randomized to receive placebo or colchicine (1.0 mg twice daily for the first day followed by a maintenance dose of 0.5 mg twice daily for 1 month in patients ≥70 kg, and halved doses for patients, 70 kg or intolerant to the highest dose) on the third post-operative day. (See, Imazio et al., Eur. Heart J., 31:2749-2754, 2010).

In another study, the effectiveness of colchicine has been shown for cardiovascular disease. In this clinical trial with a prospective, randomized, observer-blinded endpoint design, 532 patients with stable coronary disease receiving aspirin and/or clopidogrel (93%) and statins (95%) were randomly assigned colchicine 0.5 mg/day or no colchicine and followed for a median of 3 years. (See, Nidorf et al., JACC, 61(4):404-410, 2013). This study showed that colchicine administered 0.5 mg/day in addition to statins and other standard secondary prevention therapies appeared to be effective for the prevention of cardiovascular events in patients with stable coronary disease (SCD).

For the treatment of gout, the recommended dose of colchicine (COLCRYS®) is 1.8 mg/day in one or multiple doses in one hour. For adults with gout, treatment is typically initiated with a dose of 1.2 mg at the first sign of symptoms followed by 0.6 mg one hour later. (Physician's Desk Reference, 68th ed., 2014).

COLCRYS® is an immediate release formulation of colchicine. Adverse effects associated with the administration of COLCRYS® include, but are not limited to, nausea, vomiting, abdominal pain, diarrhea, hair loss, weakness, nerve irritation, severe anemia, low white blood counts, and low platelets. (See, Physician's Desk Reference, 68th ed., 2014).

The presently described methods and compositions address these and other needs by providing a modified formulation of colchicine characterized by sustained release of the active ingredient for once-daily administration. This formulation is designed not only to improve patient compliance and mitigate side effects commonly associated with higher or immediate-release doses, but also to achieve a therapeutic profile suitable for long-term management of chronic inflammatory conditions. Importantly, the sustained-release formulation enables consistent systemic exposure to colchicine at levels effective to suppress aberrant activation of inflammatory pathways, such as the NOD-, LRR- and pyrin domain-containing protein 3 (NLRP3) inflammasome, and to reduce circulating pro-inflammatory cytokines including IL-1β, IL-6, and TNF-α. The presently described methods and compositions are particularly effective in management, treatment, and/or prevention of persistent systemic inflammation arising after viral infection or vaccination, such as chronic conditions including chronic inflammation following Coronavirus disease (COVID)-19 immunization and/or infection. By targeting the innate immune mechanisms that drive chronic inflammation, the presently described methods and compositions offer an important and as yet unexplored disease-modifying approach to improving symptoms, reducing inflammatory biomarkers, and preventing or ameliorating downstream complications associated with prolonged immune activation.

### BRIEF SUMMARY OF THE INVENTION

According to aspects of the presently described methods and compositions illustrated herein, there are provided methods of treating chronic inflammation, for instance in a human subject, with such methods comprising administering to the subject in need thereof a daily oral dose of about 0.5 mg of colchicine, or a pharmaceutically acceptable salt thereof, wherein the subject exhibits signs or biomarkers of persistent systemic inflammation, and wherein said administration reduces the levels of inflammatory biomarkers in said subject, said biomarkers selected from high-sensitivity C-reactive protein (hs-CRP), interleukin-1β (IL-1β), interleukin-6 (IL-6), tumor necrosis factor-alpha (TNF-α), and myeloperoxidase (MPO).

According to certain embodiments described herein, the subject exhibits elevated levels of hs-CRP, for instance, levels that are above 2 mg/L of the baseline level.

According to other embodiments described herein, the subject exhibits elevated IL-6, for instance, levels that are above 3 μg/mL of baseline levels.

According to other embodiments, the subject presents with clinical symptoms associated with chronic inflammation, selected from fatigue, joint pain, myalgia, cognitive impairment, and low-grade fever.

According to additional embodiments disclosed herein, colchicine is administered once per day for a duration of at least 3 months.

According to certain embodiments of the described methods, the duration of treatment is extended to at least 12 months for maintenance of reduced inflammatory biomarkers.

According to additional embodiments, colchicine is administered in combination with one or more additional anti-inflammatory agents selected from non-steroidal anti-inflammatory drugs (NSAIDs), corticosteroids, and interleukin-6 inhibitors.

According to some embodiments, the subject is monitored periodically for inflammatory biomarker levels through blood-based assays at intervals of 4 to 12 weeks.

According to other embodiments, administration of colchicine results in a reduction of hs-CRP levels of at least 30% from baseline after about 8 weeks of treatment.

According to embodiments, colchicine is administered in a formulation, such as, immediate-release tablets, extended-release tablets, and oral suspensions.

According to other embodiments, the subject's inflammatory biomarker levels and/or other inflammatory symptoms have previously failed to respond to treatment with NSAIDs or corticosteroids.

According to various described embodiments, treatment reduces the frequency or severity of flares in an inflammatory condition, where the inflammatory condition is, for example, rheumatoid arthritis, systemic lupus erythematosus, and inflammatory bowel disease. According to other embodiments, colchicine is co-administered with another active agent, such as one or more statins, where the one or more statin is one or more of atorvastatin, rosuvastatin, simvastatin, pravastatin, and lovastatin, or a pharmaceutically acceptable salt thereof.

According to various embodiments described herein, the administration of colchicine reduces inflammatory biomarkers in the subject such as hs-CRP, IL-6, and MPO.

According to aspects of the methods illustrated herein, there are provided methods of treating chronic inflammation in a human subject following COVID-19 vaccination and/or infection, the method comprising administering to the subject a daily oral dose of about 0.5 mg of colchicine, or a pharmaceutically acceptable salt thereof, wherein the subject exhibits persistent systemic inflammation that arises within 12 weeks of receiving a COVID-19 vaccine and is characterized by elevated levels of one or more inflammatory biomarkers, such as, hs-CRP, IL-1β, IL-6, TNF-α, and MPO, and wherein said treatment reduces the levels of said inflammatory biomarkers.

According to certain embodiments of the described methods, the COVID-19 vaccine is an mRNA-based vaccine.

According to embodiments described herein, the COVID-19 vaccine is one or more of BNT162b2 (Pfizer-BioNTech), mRNA-1273 (Moderna), and other vaccines comprising lipid nanoparticle-encapsulated mRNA.

According to certain embodiments, the subject developed myocarditis, pericarditis, or myopericarditis after vaccination, and colchicine is administered to reduce cardiac inflammation.

According to certain embodiments of the methods described herein, colchicine administration is initiated within about 3 months of the onset of chronic inflammatory symptoms in the subject post-vaccination.

According to other embodiments of the methods described herein, the subject exhibits clinical symptoms of chronic inflammation such as fatigue, arthralgia, myalgia, chest discomfort, shortness of breath, and/or cognitive impairment.

According to one embodiment, colchicine treatment is continued for a period of at least about 3 months.

According to certain embodiments, the subject exhibits a reduction in hs-CRP of at least 30% from baseline after 8 weeks of treatment with colchicine.

According to other embodiments, the subject has previously failed to respond to corticosteroids or NSAIDs for reduction in or amelioration of post-vaccination symptoms of inflammation.

According to one or more embodiments, colchicine is administered in combination with one or more immunomodulatory agents such as corticosteroids, interleukin-1 inhibitors, and interleukin-6 inhibitors.

According to other embodiments, the presence or absence of inflammation is confirmed by imaging of affected organs or tissues, including cardiac MRI for myocarditis or PET-CT for systemic inflammation.

According to certain embodiments, the subject is monitored periodically through blood-based assays of hs-CRP, IL-1 and IL-6 during and/or after the course of treatment.

### BRIEF DESCRIPTION OF THE DRAWINGS/FIGURES

The patent or application file contains at least one drawing executed in color. Copies of this patent or patent application publication with color drawings will be provided by the Office upon request and payment of the necessary fee.

FIG. 1 shows the dissolution profiles for colchicine sustained-release formulations containing 10%, 15% and 20%, respectively, of an exemplary retarding agent.

FIG. 2 shows the dissolution profiles for colchicine sustained-release formulation containing 30% of an exemplary retarding agent and tablet hardnesses of 50N and 130N, respectively.

FIG. 3 shows the dissolution profiles for colchicine sustained-release formulations according to FIGS. 1 and 2.

FIG. 4 shows the dissolution profile for a colchicine sustained-release formulation containing 0% of an exemplary retarding agent.

FIG. 5 shows the dissolution profile for a colchicine sustained-release formulation containing 25% of an exemplary retarding agent.

FIG. 6 shows the dissolution profile for colchicine formulation containing 0%, 23.3%, 26.6% and 30% of an exemplary retarding agent.

FIGS. 7A, 7B, 7C and 7D show plasma colchicine levels (ng/ml) as a function of time (hrs) for colchicine formulations according to FIG. 6.

FIG. 8 shows plasma colchicine levels (ng/mL) as a function of time (hrs) for colchicine formulations according to FIG. 6.

FIG. 9 shows the levels of colchicine in neutrophils on Day 1 and Day 10 for colchicine formulations according to FIG. 6.

### DETAILED DESCRIPTION OF THE INVENTION

### I. Definitions

For the purposes of methods and compositions describes herein, the term “colchicine” includes colchicine and/or any pharmaceutically acceptable salts thereof, and/or any combinations thereof.

“Pharmaceutically acceptable” means that which is generally safe, non-toxic, and neither biologically nor otherwise undesirable and includes that which is acceptable for veterinary use as well as human pharmaceutical use.

“Pharmaceutically acceptable salts” includes derivatives of colchicine, wherein the colchicine is modified by making acid or base addition salts thereof, and further refers to pharmaceutically acceptable solvates, including hydrates, and co-crystals of such compounds and such salts. Examples of pharmaceutically acceptable salts include, but are not limited to, mineral or organic acid addition salts of basic residues such as amines; alkali or organic addition salts of acidic residues; and the like, and combinations comprising one or more of the foregoing salts. The pharmaceutically acceptable salts include non-toxic salts and the quaternary ammonium salts of the colchicine. For example, non-toxic acid salts include those derived from inorganic acids such as hydrochloric, hydrobromic, sulfuric, sulfamic, phosphoric, nitric and the like; other acceptable inorganic salts include metal salts such as sodium salt, potassium salt, cesium salt, and the like; and alkaline earth metal salts, such as calcium salt, magnesium salt, and the like, and combinations comprising one or more of the foregoing salts. Pharmaceutically acceptable organic salts includes salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH where the value of n is a whole integer between 0 and 4, and the like; organic amine salts such as triethylamine salt, pyridine salt, picoline salt, ethanolamine salt, triethanolamine salt, dicyclohexylamine salt, N,N′-dibenzylethylenediamine salt, and the like; and amino acid salts such as arginate, asparaginate, glutamate, and the like; and combinations comprising one or more of the foregoing salts; organic amine salts such as triethylamine salt, pyridine salt, picoline salt, ethanolamine salt, triethanolamine salt, dicyclohexylamine salt, N,N′-dibenzylethylenediamine salt, and the like; and amino acid salts such as arginate, asparaginate, glutamate, and the like; and combinations comprising one or more of the foregoing salts. All forms of such derivatives of colchicine are contemplated herein, including all crystalline, amorphous, and polymorph forms. Specific colchicine salts include colchicine hydrochloride, colchicine dihydrochloride, and co-crystals, hydrates or solvates thereof.

“Pharmacokinetic parameters” describe the in vivo characteristics of an active agent (or a metabolite or a surrogate marker for the active agent) over time, such as plasma concentration (C), Cₘₐₓ, Cₙ, C₂₄, Tₘₐₓ, and AUC. “Cₘₐₓ” is the measured plasma concentration of the active agent at the point of maximum, or peak, concentration. “Cₘᵢₙ” is the measured plasma concentration of the active agent at the point of minimum concentration. “Cₙ” is the measured plasma concentration of the active agent at about n hours after administration. “C₂₄” is the measured plasma concentration of the active agent at about 24 hours after administration. The term “Tₘₐₓ” refers to the time at which the measured plasma concentration of the active agent is the highest after administration of the active agent. “AUC” is the area under the curve of a graph of the measured plasma concentration of an active agent vs. time, measured from one time point to another time point. For example, AUC₀₋ₜ is the area under the curve of plasma concentration versus time from time 0 to time t, where t can be the last time point with measurable plasma concentration for an individual formulation. The AUC_{0-∞} or AUC_{0-INF} is the calculated area under the curve of plasma concentration versus time from time 0 to time infinity. In steady-state studies, AUC₀₋ₜ is the area under the curve of plasma concentration over the dosing interval (i.e., from time 0 to time τ (tau), where tau is the length of the dosing interval. Other pharmacokinetic parameters are the parameter Ke or Kel, the terminal elimination rate constant calculated from a semi-log plot of the plasma concentration versus time curve; t_{1/2} the terminal elimination half-life, calculated as 0.693/Kel; CL/F denotes the apparent total body clearance after administration, calculated as Total Dose/Total AUC_{∞}; and Varea/F denotes the apparent total volume of distribution after administration, calculated as Total Dose/(Total AUC_{∞}×Kel).

“Efficacy” means the ability of an active agent administered to a patient or subject to produce a therapeutic effect in the patient.

“Bioavailability” means the extent or rate at which an active agent is absorbed into a living system or is made available at the site of physiological activity. For active agents that are intended to be absorbed into the bloodstream, bioavailability data for a given formulation may provide an estimate of the relative fraction of the administered dose that is absorbed into the systemic circulation. “Bioavailability” can be characterized by one or more pharmacokinetic parameters.

A “dosage form” means a unit of administration of an active agent. Examples of dosage forms include tablets, capsules, injections, suspensions, liquids, emulsions, creams, ointments, suppositories, inhalable forms, transdermal forms, and the like.

An “immediate release formulation” refers to a formulation that releases greater than or equal to about 80% of the pharmaceutical agent in less than or equal to about 30 min.

For the purposes of this application, an enhancing agent (“enhancer”) is defined as any non-pharmaceutically active ingredient that improves the therapeutic potential of a formulation.

“Sustained release” is defined herein as release of a pharmaceutical agent in a continuous manner over a prolonged period of time.

By “prolonged period of time” it is meant a continuous period of time of greater than about 1 hour, greater than about 4 hours, greater than about 8 hours, greater than about 12 hours, greater than about 16 hours, or up to more than about 24 hours.

As used herein, unless otherwise noted, “rate of release” or “release rate” or “dissolution rate” of a drug refers to the quantity of drug released from a dosage form per unit time, e.g., milligrams of drug released per hour (mg/hr) or a percentage of a total drug dose released per hour. Drug release rates for dosage forms are typically measured as an in vitro rate of drug release, i.e., a quantity of drug released from the dosage form per unit time measured under appropriate conditions and in a suitable fluid. The release rates referred to herein are determined by placing a dosage form to be tested in a medium in an appropriate dissolution bath. Aliquots of the medium, collected at pre-set intervals, are then injected into a chromatographic system fitted with an appropriate detector to quantify the amounts of drug released during the testing intervals.

Side effect is defined herein as a secondary and usually adverse effect of a drug.

As used herein, the term “about” or “approximately” refers to a variation of 10% from the indicated values (e.g., 50%, 45%, 40%, etc.), or in case of a range of values, means a 10% variation from both the lower and upper limits of such ranges. For instance, “about 50%” refers to a range of between 45% and 55%.

Terms such as “treating” or “treatment” or “to treat” or “alleviating” or “to alleviate” refer to both: 1) therapeutic measures that cure, slow down, lessen symptoms of, reverse, and/or halt progression of a diagnosed pathologic condition or disorder, and 2) prophylactic or preventative measures that prevent and/or slow and/or ameliorate the development of a targeted pathologic condition or disorder. Thus, those in need of treatment include those already with the disorder; those prone to have the disorder; and those in whom the disorder is to be prevented. Beneficial or desired clinical results include, but are not limited to, alleviation or amelioration of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

As used herein, the term “persistent” means continuous over an extended or prolonged period of time, relative to the life of the subject, or relative to the typical course of the clinical condition indicated, such as viral infection, etc. Persistent means, in some embodiments, lasting longer than the normal or usual course of action for a disease or clinical condition, even after attempts have been made to treat the condition or disease. In some embodiments, persistent means continuous or incessant for a period of at least 2, 3, 4, 5, 6, 7, 8, 9, 10, 12, 14, 16, 18, 20, 30, 40, 50, or more weeks. Persistent in other embodiments means continuously present for a period of a year or more, such as for 2, 3, 4, 5, 6, 8, 10, or even 15 years or more.

The term “periodic” or “periodically” especially in the context of following progress of recovery from clinical conditions after treatment by way of the presently described methods means, in some embodiments, tracking the condition's status by blood test at least once per day, twice per day, three times per day, or in some embodiments, tracking the blood markers continuously. Such blood markers include those described herein characteristic of inflammatory markers, such as TNFα, IL-1, IL-6, and the like. Periodically, in some embodiments, means once or more per day, for several days, such as for at least 1, 2, 3, 4, 5, 6, 7, 14, 21, 28, 35, or more days, or even one or more months, such as for 1, 2, 3, 4, 5, 6, 8, 10, or even 12 months, on a monthly or weekly basis. In general, periodically means tracking the course of the recovery of the disease post treatment at regular intervals sufficient to measure or detect a clinical change in condition, such as the increase or decrease in the protein or mRNA level of the aforementioned inflammatory markers in the subject's blood.

By “subject” or “individual” or “animal” or “patient” or “mammal,” is meant any subject, particularly a mammalian subject, for whom diagnosis, prognosis, or therapy is desired. Mammalian subjects include humans, domestic animals, farm animals, and zoo, sports, or pet animals such as dogs, cats, guinea pigs, rabbits, rats, mice, horses, cattle, cows, bears, and so on. The meaning of the terms “eukaryote,” “animal,” “mammal,” and the like are well known in the art and can, for example, be deduced from Wehner und Gehring (1995; Thieme Verlag). In the context of the present disclosure, it is also envisaged that animals are to be treated which are economically, agronomically or scientifically important. Scientifically important organisms include, but are not limited to, mice, rats, monkeys, dogs, cats, and rabbits. Non-limiting examples of agronomically important animals are sheep, cattle, horses, ostrich, turkeys, and pigs, while, for example, cats and dogs may be considered as economically, domestically, and scientifically important animals. In one embodiment, the subject/patient is a mammal; in another embodiment, the subject/patient is a human or a non-human mammal (such as, e.g., a guinea pig, a hamster, a rat, a mouse, a rabbit, a dog, a cat, a horse, a monkey, an ape, a marmoset, a baboon, a gorilla, a chimpanzee, an orangutan, a gibbon, a sheep, cattle, or a pig); most preferably, the subject/patient is a human.

### II. Colchicine

In the following, colchicine used according to the present invention will be described in detail. The chemical structure of colchicine (ChemID 2012) is as follows:

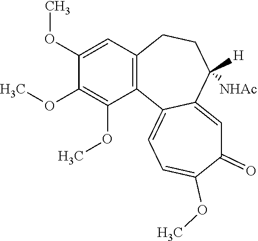 [SMILES: COc1cc2c(c(OC)c1OC)-c1ccc(OC)c(=O)cc1[C@@H](NC(C)=O)CC2]

The chemical name of colchicine is: N [5,6,7,9-tetrahydro-1,2,3,10-tetratmethoxy 9-oxobenzo[a]heptalen-7-yl], (S)-acetamide; molecular formula: C₂₂H₂₅NO₆; CAS number: 64-86-8.

Colchicine is an anti-inflammatory drug with a long history in human medicine, used for the symptomatic treatment of inflammatory diseases, most prominently gout. It is a natural product which can be extracted from two plants of the lily family, Colchicum autumnale and Gloriosa superba. Colchicine is a tricyclic alkaloid and has a molecular mass of 399.437. The active ingredient colchicine as well as its tablet formulation is listed in various national and international pharmacopeias such as the United States Pharmacopeia (USP).

The positive effect of its plant source in the treatment of rheumatism and swelling was described first already around 1500 B.C. in Egypt. Its use in gout was first described around 1500 years ago. (See, Graham and Roberts, Ann. Rheum. Dis., 12(1):16-9, 1953). Today, the therapeutic value of colchicine is well established in a number of inflammatory diseases and approved by FDA for the prophylaxis and treatment of acute gout flares and familial Mediterranean fever (FMF). Other important established, though off-label uses are amongst others, Behçet's disease and recurrent pericarditis. In all known indications, it is generally administered orally as solid tablets in strengths of 0.5-0.6 mg/tablet (e.g. Europe and United States, respectively). The pharmacotherapeutic mechanism of action of colchicine in diverse disorders is not fully understood, though it is known that the drug accumulates preferentially in leucocytes, particularly neutrophils which is important for its therapeutic effect. Three major interactions of colchicine with specific proteins modulate its pharmacokinetics: tubulin, cytochrome P450 3A4 (CYP3A4), and P-glycoprotein. It is assumed that most therapeutic effects of the drug are related to its capacity to bind to β-tubulin, thus inhibiting self-assembly and polymerization of microtubules. Availability of tubulin is essential for several cellular functions such as mitosis. Therefore, colchicine effectively functions as a “mitotic poison” or spindle poison. By inhibiting microtubule self-assembly, colchicine interferes with many cellular functions involved in the immune response such as modulation of the production of chemokines chemokines and prostanoids and inhibition of neutrophil and endothelial cell adhesion molecules. Eventually it decreases neutrophil degranulation, chemotaxis and phagocytosis, thus reducing the initiation and amplification of inflammation. Colchicine also inhibits uric acid crystal deposition (a process important to the genesis of gout), which is enhanced by a low pH in the tissues, probably by inhibiting oxidation of glucose and subsequent lactic acid reduction in leukocytes. (See, Imazio et al., Eur. Heart J., 30(5):532-9, 2009; Cocco et al., Eur. J. Intern. Med., 21(6):503-8, 2010; and Stanton et al., Med. Res. Rev., 31(3):443-81, 2011). In the management of pericarditis, colchicine exerts its therapeutic effect by suppressing acute pericardial inflammation. However, the exact cellular and molecular mechanisms of how colchicine relieves pain and inflammation in acute pericarditis and prevents recurrences are not fully understood.

Colchicine, in the context of the present disclosure, is administered to subjects in need thereof for the prevention and/or treatment of cardiovascular diseases and/or inflammatory diseases.

### III. Sustained Release Formulations

The present disclosure additionally provides description of a sustained release colchicine formulation for the treatment or prevention of a cardiovascular disease and/or an inflammatory disorder in a subject, wherein colchicine is released from the formulation at a sustained rate along a pre-determined or desired release profile. Such release is achieved by incorporation into the formulation of an extended-release component and an optional immediate-release component. The colchicine formulations described herein are, for instance, formulated in a dosage form selected from a tablet, a pill, a capsule, a caplet, a troche, a sachet, a cachet, a pouch, sprinkles, or any other form suitable for oral administration.

According to the present disclosure, colchicine as described herein (i.e., inter alia, in the form of a (pharmaceutical) composition) is administered in the form of a sustained release preparation. Other expressions like “extended release,” “controlled release,” “modified release,” or “delayed release,” “preparation,” or “formulation,” are understood herein to have the same meaning as “sustained release preparation.” Such preparations are in any form conceivable to the skilled person and include, for example, pharmaceutical forms for oral (solid, semi-solid, liquid), dermal (dermal patch), sublingual, parenteral (injection), ophthalmic (eye drops, gel or ointment), or rectal (suppository) administration, as long as a sustained release of the active agent is ensured.

In accordance with various embodiments described herein, sustained release preparations encompass all pharmaceutical forms that create a steady drug release profile making the drug substance available over an extended period of time following application to the patient. Such an extended period of time is, for example, between 10, 20, 30, 40, 50, or 60 minutes and about 2, 3, 4, 5, 6, 7, 8, 9, 10, 11, 12, 13, 14, 15, 16, 17, 18, 19, 20, 21, 22, 23, or 24 hours. Extended release is, in some embodiments, defined functionally as the release of over 50, 55, 60, 65, 70, 75, 80, 85, 90, 95, or 99 percent (%) of colchicine after about 10, 20, 30, 40, 50, or 60 minutes and about 2, 3, 4, 5, 6, 7, 8, 9, 10, 11, 12, 13, 14, 15, 16, 17, 18, 19, 20, 21, 22, 23, or 24 hours. The phrase, “extended release,” as used herein, in some embodiments, is defined as making colchicine available to the patient/subject regardless of uptake, as some colchicine is typically never be absorbed by the patient. Various extended-release dosage forms are able to be designed readily by one of skill in art as disclosed herein to achieve delivery and sustained release of colchicine to the liver and/or both the small and large intestines, to only the small intestine, or to only the large intestine.

In some embodiments, sustained release preparations are pH independent. This allows such preparations to dissolve in almost any environment. In other embodiments, sustained release preparations are pH dependent. This allows release to be achieved at some generally predictable location in the lower intestinal tract more distal to that which would have been accomplished if there had been no delayed release alterations. A method for delay of release is, e.g., a coating. Any coatings are generally applied to a sufficient thickness such that the entire coating does not dissolve in the gastrointestinal fluids at pH below about 5, but does dissolve at pH about 5 and above. It is envisioned that any anionic polymer exhibiting a pH-dependent solubility profile is useful as an enteric coating in the practice of the present methods and compositions to achieve delivery to the lower gastrointestinal tract. Polymers and compatible mixtures thereof are employed to provide the coating for the delayed or the extended release of active ingredients, and some of their properties, include, but are not limited to: shellac, also called purified lac, a refined product obtained from the resinous secretion of an insect. This coating dissolves in media of pH>7.

In some embodiments, sustained release preparations are influenced by the presence of alcohol in the body. The presence of alcohol is a patient's body can increase dissolution of the composition and can cause immediate release of the entire dose. This effect is known as “dose dumping” and is dependent on the alcohol solubility of the materials. For sustained release preparations that contain a higher dose for slow release over 24 hours, for instance, this effect can have safety concerns and can even be life threatening.

To achieve a uniform or continuous rate of release, in certain embodiments, sustained release preparations are prepared by incorporating time-release hydrophilic matrices. These time-release hydrophilic matrices are known in the field of drug formulations. For example, one such hydrophilic matrix is hydroxypropyl methylcellulose (HPMC) or hypromellose (commercially also available under the names METHOCEL®, METOLOSE®, PHARMACOAT®, and QUALI-VR). Hydrophilic matrices provide an initial release of the drug product in the initial phase mainly triggered by a rapid swelling of the surface of the matrix tablet, combined with an erosion process leading to an immediate release of the drug substance distributed close to the surface of the tablet. In an embodiment, about 50%, about 45%, about 40%, about 35%, about 30%, about 25%, about 20%, about 15%, or about 10% of the drug substance is immediately released, depending on the desired release profile. In an embodiment, at least about 20% of the drug substance is immediately released. In another embodiment, at least about 20% of the drug substance is released within about the first 30 minutes. Within the initial swelling of the tablet surface a gel formation of the hydrophilic matrix starts. This gelling prevents the tablet core from dissolving and disintegrating immediately, thereby allowing the main part of the drug substances to dissolve slowly over time within this gel structure and diffuse into solution following the rules of Fick's law. The diffusion itself may be triggered in this formulation approach by the concentration of the hypromellose and the viscosity of the formed gel, defined over the molecular weight of the hypromellose. Therefore, drug release profiles is, in some embodiments, modified by varying different viscosity grades of hypromellose or mixtures thereof. All corresponding formulation and process parameters achieving the predicted release profile are common knowledge and is adjustable using actual development technologies e.g. formulation screenings, statistical trials designs.

In an embodiment, the substance responsible for sustained release of the controlled-release formulation is further mixed with a binder. The binder is added to increase the mechanical strength of the granules and tablets during formation. Binders are added to the formulation in different ways, including: (1) as a dry powder that is mixed with other ingredients before wet agglomeration, (2) as a solution that is used as agglomeration liquid during wet agglomeration, and is referred to as a solution binder, and (3) as a dry powder that is mixed with the other ingredients before compaction. In this form the binder is referred to as a dry binder. Solution binders are a common way of incorporating a binder into granules. In certain embodiments, the binder added to the formulation is added in the form of a dry powder binder. Non-limiting examples of binders useful for the core include hydrogenated vegetable oil, castor oil, paraffin, higher aliphatic alcohols, higher aliphatic acids, long chain fatty acids, fatty acid esters, wax-like materials such as fatty alcohols, fatty acid esters, fatty acid glycerides, hydrogenated fats, hydrocarbons, normal waxes, stearic acid, stearyl alcohol, hydrophobic and hydrophilic polymers having hydrocarbon backbones, and mixtures thereof. Specific examples of water-soluble polymer binders include modified starch, gelatin, polyvinylpyrrolidone, cellulose derivatives (such as, for example, hydroxypropyl methylcellulose (HPMC) and hydroxypropyl cellulose (HPC)), polyvinyl alcohol, and mixtures thereof. In an embodiment, the binder is HPMC. In another embodiment, the binder is hypromellose having a viscosity of 6 mPa*s. In an embodiment, the binder is present in an amount of from about 1% to about 30% by weight of the formulation.

In another embodiment, the sustained release formulation includes a disintegrant. A disintegrant refers to an agent used in pharmaceutical preparation of tablets, which causes them to disintegrate and release their medicinal substances on contact with moisture. In an embodiment, the disintegrant is water soluble to support the disintegration of a tablet in the stomach. Non-limiting examples of disintegrants for use in the formulation include sucrose, lactose, in particular lactose monohydrate, trehalose, maltose, mannitol and sorbitol, croscarmellose sodium, crospovidone, alginic acid, sodium alginate, methacrylic acid divinyl benzene (DVB), cross-linked polyvinyl povidone (PVP), microcrystalline cellulose, polacrilin potassium, sodium starch glycolate, starch, pregelatinized starch and mixtures thereof. In at least one embodiment the disintegrant is selected from microcrystalline cellulose (e.g. Avicel PH101), cross-linked polyvinylpyrrolidone (e.g. KOLLIDON® CL), cross-linked sodium carboxymethylcellulose (e.g. AC-DI-SOL™), starch or starch derivatives such as sodium starch glycolate (e.g. EXPLOTAB®), or combinations with starch (e.g. PRIMOJEL™), swellable ion-exchange resins, such as AMBERLITE™ IRP 88, formaldehyde-casein (e.g. ESMA SPRENG™), and mixtures thereof.

In another embodiment, the sustained release formulation includes a filling agent or filler. A filling agent refers to an inert substance that creates certain desired bulk, flow properties, and compression characteristics helpful in the preparation of tablets. Non-limiting examples of filling agents for incorporation into the formulation include sucrose, lactose, in particular lactose monohydrate, trehalose, maltose, mannitol and sorbitol, croscarmellose sodium, crospovidone, alginic acid, sodium alginate, methacrylic acid DVB, cross-linked PVP, microcrystalline cellulose, polacrilin potassium, sodium starch glycolate, starch, pregelatinized starch and mixtures thereof. In an embodiment, lactose monohydrate is included as a filling agent in an amount of about 10% to about 80%, preferably about 59%, by weight of the tablet. In an embodiment, pregelatinized starch is included as a filling agent in an amount of about 5% to about 50%, preferably about 7.5%, by weight of the tablet.

In another embodiment, the sustained release formulation includes a release retarding agent for maintaining a uniform release rate of the drug. Examples of retarding agents include, but are not limited to, cellulose ethers, cellulose esters, acrylic acid copolymers, waxes, gums, glyceryl fatty acid esters and sucrose fatty acid esters. In one embodiment, the retarding agent is RETALAC® (Meggle), a spray agglomerated blend of 50 parts lactose monohydrate and 50 parts hypromellose. The viscosity of hypromellose used herein may range from 6 mPa*s-100,000 mPa*s. In an embodiment, the viscosity of hypromellose used is 4000 mPa*s. Adjusting the amount of retarding agent in the composition may alter the release rate of the drug. In one embodiment, the retarding agent of the formulation of the present invention releases colchicine in a continuous and uniform manner and is adjusted in such a way that about 80% of the active ingredient is released in vitro in the predetermined period of time. By way of example, and by no means limiting the scope of the invention, the period of time may be not more than 24 hours, not more than 16 hours, not more than 12 hours, not more than 8 hours, not more than 6 hours, not more than 4 hours, not more than 3.5 hours, or not more than 1.5 hours depending on desired attributes of the final product. It is understood that the release rate can vary based on whether the experiment is conducted in vitro or in vivo. Therefore, if the desired release rate is between about 1.5 to about 3.5 hours in vitro or between about 1.5 to about 6 hours in vitro, the release rate under in vivo conditions, depending on the experimental conditions, may actually be different. In an embodiment, the sustained release formulation releases colchicine in a continuous and uniform manner in such a way that about 80% of the active ingredient is released in vitro in between about 1.5 and about 3.5 hours.

In another embodiment, the sustained release formulation includes a glidant. A glidant is used typically to improve powder flow properties prior to and during tableting and to reduce caking. Suitable glidants include, but are not limited to, colloidal silicon dioxide, magnesium trisilicate, powdered cellulose, talc, tribasic calcium phosphate, and the like. In one embodiment, talc is included as a glidant in an amount of about 0.05% to about 5%, preferably about 1%, by weight of the tablet.

In another embodiment, the sustained release formulation includes a lubricant. Lubricants are typically added to pharmaceutical formulations to decrease any friction that occurs between the solid and the die wall during tablet manufacturing. High friction during tableting can cause a series of problems, including inadequate tablet quality (capping or even fragmentation of tablets during ejection, and vertical scratches on tablet edges) and may even stop production. Accordingly, lubricants are added to certain tablet formulations including certain embodiments of the formulations described herein. Non-limiting examples of lubricants useful for the core include glyceryl behenate, stearic acid, hydrogenated vegetable oils (such as hydrogenated cottonseed oil (STEROTEX®), hydrogenated soybean oil (STEROTEX® HM) and hydrogenated soybean oil & castor wax (STEROTEX® K)), stearyl alcohol, leucine, polyethylene glycol (MW 1450, suitably 4000, and higher), magnesium stearate, glyceryl monostearate, polyethylene glycol, ethylene oxide polymers (for example, available under the registered trademark CARBOWAX® from Union Carbide, Inc., Danbury, Conn.), sodium lauryl sulfate, magnesium lauryl sulfate, sodium oleate, sodium stearyl fumarate, DL-leucine, colloidal silica, mixtures thereof and others as known in the art. In one embodiment, stearic acid is included as a lubricant in an amount of about 0.05% to about 5%, preferably about 1%, by weight of the tablet.

In some embodiments, sweeteners are added to the described formulations as a taste-masking coating of the matrix dosage forms. Including, for example, glucose (corn syrup), dextrose, invert sugar, fructose, and mixtures thereof (when not used as a carrier); saccharin and its various salts, such as sodium salt; dipeptide sweeteners such as aspartame; dihydrochalcone compounds, glycyrrhizin; Stevia rebaudiana (generic chemical name Stevioside); chloro derivatives or sucrose such as sucralose; and sugar alcohols such as sorbitol, mannitol, xylitol, and the like. Also contemplated are hydrogenated starch hydrolysates and the synthetic sweeteners such as 3,6-dihydro-6-methyl-1-1-1,2,3-oxathiazin-4-1-2,2-dioxide, particularly the potassium salt (acesulfame-K), and sodium and calcium salts thereof. The sweeteners can be used alone or in any combination thereof.

The controlled-release formulations described herein, in some embodiments, further contain one or more pharmaceutically acceptable excipients such as, but not limited to, granulating aids or agents, colorants, flavorants, pH adjusters, anti-adherents, glidants, and like excipients conventionally added to pharmaceutical compositions in the formulation process. In an embodiment, a coloring excipient is added to provide a visual change preventing abuse. It can color simultaneously the liquid or the particles or one independently of the other. Among suitable coloring excipients the following are non-limiting examples: indigotine, cochineal carminic acid, yellow orange S, allura red AC, iron oxides, curcumin, riboflavin, tartrazine, quinoline yellow, azorubine, amaranth, carmines, erythosine, red 2G, patented blue V, glittering blue FCF, chlorophylls, copper complexes of chlorophylls, green S, caramel, glittering black BN, Carbo vegetabilis, brown FK and HT, carotenoids, annatto extracts, paprika extracts, lycopene, lutein, canthaxanthin, beetroot red, anthocyanins, calcium carbonate, titanium dioxide, aluminum, silver, gold, litholrubin BK, or any other coloring excipient suitable for an oral administration.

In an embodiment, a sustained release formulation are coated. Coatings provide a variety of functions. In some embodiments, coatings are added, for example, to achieve delayed release, resistance to acid, targeted release in the lower GI tract, avoidance of bad taste in mouth. In some embodiments, coatings are added to the tablet formulation to protect the API/tablet from light and to provide for better mechanical resistance. Of course, it should be appreciated that a coating serves other functions and a person skilled in the art knows the purpose of tablet coating.

In some embodiments, pharmaceutical composition and/or the solid carrier particles described herein are coated with one or more enteric coatings, seal coatings, film coatings, barrier coatings, compress coatings, fast disintegrating coatings, or enzyme degradable coatings. Multiple coatings may be applied for desired performance. Further, one or more of the active agents are especially formulated, in certain embodiments, for immediate release, pulsatile release, controlled release, extended release, delayed release, targeted release, synchronized release, or targeted delayed release. In some embodiments, the formulation includes combinations of typical pharmaceutical actives (e.g., pseudephedrine) and vitamins (e.g., Vitamin C), minerals (Ca, Mg, Zn, K) or other supplements (e.g., St. John's Wort, echinacea, amino acids). For release/absorption control, solid carriers are, in various embodiments, made of various component types and levels or thicknesses of coats, with or without an active ingredient. Such diverse solid carriers are, in some embodiments, blended in a dosage form to achieve the desired performance. The liquid formulations are in certain embodiments delivered to, and adapted for, oral, nasal, buccal, ocular, urethral, transmucosal, vaginal, topical, or rectal delivery, for example, although oral delivery is particularly envisioned.

When formulated with microparticles or nanoparticles, the drug release profile is adapted by adding a coating, e.g., a hard or soft gelatin coating, a starch coating, a resin or polymer coating, and/or a cellulosic coating, for example. Although not limited to microparticles or nanoparticles (as in, e.g., microcapsules or nanocapsules), such dosage forms are further coated with, in some embodiments, for example, a seal coating, an enteric coating, an extended-release coating, or a targeted delayed release coating. The term “enteric coating” as used herein relates to a mixture of pharmaceutically acceptable excipients that are applied to, combined with, mixed with, or otherwise added to the carrier or composition. In a certain embodiment, the coating is applied to an active pharmaceutical ingredient (API), such as colchicine, that is compressed, molded or extruded and also includes, in certain embodiments: gelatin, and/or pellets, beads, granules, or particles of the carrier or composition. The coating is, in certain embodiments, applied through an aqueous dispersion or after dissolving in appropriate solvent. In some embodiments, the carrier is fully or partially biodegradable.

In an embodiment, polymethacrylate acrylic polymers are employed as coating polymers. In at least one embodiment, the coating is an acrylic resin lacquer used in the form of an aqueous dispersion, such as that which is commercially available from Rohm Pharma under the trade name EUDRAGIT® or from BASF under the trade name KOLLICOAT®. In other embodiments, EUDRAGIT® E100 is added as the coating polymer, which is a cationic copolymer based on dimethylaminoethyl methacrylate and neutral methacrylic esters having a average molecular weight is approximately 150,000. Different coating polymers of the various embodiments described herein are, in some embodiments, mixed together in a desired ratio to obtain a coating that possesses the desired drug dissolution profile. Coating methods include, for example, spraying a solution of the polymer on the tablets, either in a pan coater or a fluid bed coating apparatus. The solvent is either organic or aqueous, depending on the nature of the polymer applied. In one embodiment, the solvent is alcohol. Tablet coating methods, in general, are well known in the art.

The compositions described herein are, in some embodiments, formulated as enteric coated delayed release oral dosage forms, i.e., as an oral dosage form of a pharmaceutical composition as described herein that includes an enteric coating to effect release in the lower gastrointestinal tract. The enteric coated dosage form will, in some embodiments, include microparticles, microgranules, micropellets, or microbeads of the active ingredient and/or other composition components, which are themselves optionally coated. The enteric coated oral dosage form is, in certain embodiments, a capsule (coated or uncoated) containing pellets, beads, or granules of the solid carrier or the composition, which are themselves optionally coated.

Carriers that are incorporated into the described formulations, in certain embodiments, include, for example, permeable and semipermeable matrices or polymers that control the release characteristics of the formulation. Such polymers include, for example, cellulose acylates, acetates, and other semi-permeable polymers such as those described in U.S. Pat. No. 4,285,987 (hereby incorporated by reference), as well as the selectively permeable polymers formed by the coprecipitation of a polycation and a polyanion as disclosed in U.S. Pat. Nos. 3,173,876; 3,276,586; 3,541,005; 3,541,006; and 3,546,142 (relevant portions of which are incorporated herein by reference for all purposes).

Other carriers that are optionally added to the described formulations include, e.g., starch, modified starch, and starch derivatives, gums, including but not limited to xanthan gum, alginic acid, other alginates, bentonite, VEEGUM® (magnesium aluminum silicate), agar, guar, locust bean gum, gum arabic, quince psyllium, flax seed, okra gum, arabinogalactan, pectin, tragacanth, scleroglucan, dextran, amylose, amylopectin, dextrin, etc., cross-linked polyvinylpyrrolidone (PVP), ion-exchange resins, such as potassium polymethacrylate, carrageenan (and derivatives), gum karaya, biosynthetic gum, etc. Other useful polymers include: polycarbonates (linear polyesters of carbonic acid); microporous materials (bisphenol, a microporous poly(vinylchloride) (PVC), micro-porous polyamides, microporous modacrylic copolymers, microporous styrene-acrylic and its copolymers); porous polysulfones, halogenated poly(vinylidene), polychloroethers, acetal polymers, polyesters prepared by esterification of a dicarboxylic acid or anhydride with an alkylene polyol, poly(alkylenesulfides), phenolics, polyesters, asymmetric porous polymers, cross-linked olefin polymers, hydrophilic microporous homopolymers, copolymers or interpolymers having a reduced bulk density, and other similar materials, poly(urethane), cross-linked chain-extended poly(urethane), poly(imides), poly(benzimidazoles), collodion, regenerated proteins, and semi-solid cross-linked poly(vinylpyrrolidone), for example.

Additional additives and their levels, and selection of a primary coating material or materials will depend on the following properties, for example: pH levels at target site (or tissue target), desirability to make tablet pH dependent or pH independent, solubility in alcohol, resistance to dissolution and disintegration in the stomach; impermeability to gastric fluids and drug/carrier/enzyme while in the stomach; ability to dissolve or disintegrate rapidly at the target intestine site; physical and chemical stability during storage; non-toxicity; easy application as a coating (substrate friendly); and economical practicality.

Further to the above, various formulations, not limiting the scope of the described formulations, illustrating the methods and formulations are described hereafter. A controlled-release tablet or capsule or the like comprises colchicine as a core coated with an immediate release layer, in one embodiment. A controlled-release double layer tablet or capsule or the like comprises a layer of sustained release and a layer of immediate release, in another embodiment. A controlled-release tablet with more than two layers comprises: (i) one or two more layers of substance controlling the sustained release, and (ii) one or two more layers of immediate release.

According to one embodiment, the composition comprising colchicine is further coated with at least one release-slowing intermediate layer of slightly soluble intermediate layer to control release of colchicine.

Traditionally, colchicine immediate release dosage forms (mostly tablets, also injections or oral solutions) have been used in the treatment of gout or FMF. Worldwide, all approved pharmaceuticals containing colchicine are approved only for gout and/or FMF and are immediate release tablets. Colchicine is also used in the prevention of certain other inflammatory diseases such as pericarditis, PPS and, most recently, patients with stable coronary heart diseases. The difference between treatment and prevention with regard to colchicine is that in treatment, an overt disease and/or ongoing inflammation is identified in the subject as presently existing in a clinical sense and needs to be addressed by some action, which in the present disclosure, is administration of one or more of the active agents or formulations described herein, for the purpose of ameliorating or lessening the symptoms of the present clinically diagnosed condition. Thus, high levels of colchicine are often required when seeking to treat a present clinical condition, which usually goes hand in hand with, or leads directly to, unwanted side effects, most prominently gastrointestinal insults, as well as increased risk of toxicity. In prevention, one does not have to suppress ongoing inflammation but rather suppress an outbreak of inflammation. Thus, supposedly lower and steadier levels of colchicine are required and are beneficial. As described herein, this is achieved by administering colchicine formulated as a sustained release preparation, as described above.

In the treatment of an acute inflammatory disease such as FMF or gout, high doses and high serum levels of colchicine are necessary and desired to suppress inflammation. Therefore, conventional tablets with a fast release and a rather low plasma half-life are suitable, but such high doses come with the negative side effects for which colchicine is known. However, in case of prevention of a cardiovascular disease, lower levels may be sufficient to inhibit neutrophil activity. The sustained release system facilitates more steady levels of colchicine and reduces the incidence of adverse events.

An advantage of colchicine administered as sustained release is, e.g., a flattening of the serum level curve (lower but broader peak levels) reduces the incidence of serious adverse events related to colchicine toxicity, also in case of potential drug interactions thereby increasing compliance. Without wishing to be bound by any specific theory, much of colchicine related toxicity is believed to stem from the fact that one or both of the excretion pathways (liver and kidney) is reduced in its activity, either by other drugs or by a disease (e.g. kidney insufficiency). In the case of a slower and extended drug absorption (extended release), the body has more time to excrete the colchicine from the system. In this case, it is less likely that colchicine levels reach toxic levels in case of defective excretion (due to drug interaction or disease). Another potential advantage of colchicine administered as a sustained release formulation is that plasma levels remain more evenly distributed (i.e., the variability of plasma levels, such as the differences in Cₘₐₓ, Tₘₐₓ, AUC or other pharmacokinetic parameters, among patients is reduced), resulting in fewer “non-responders” to the treatment. In addition, administration of colchicine as sustained release is resistant to dose dumping, therefore the dissolution of the composition is not significantly influenced by alcohol.

Further, sustaining the release of colchicine expands the time where colchicine is present in the blood in therapeutic levels. This results in a more efficient inhibition of disease progression and, thus, improving the clinical outcome.

Furthermore, for the prophylactic uses described herein, colchicine does not have to travel deep into the subject's tissue (like for gout), and instead colchicine may be active in the blood system directly (in the vessels) where it acts on the plaques and especially on inflammatory blood cells (such as neutrophils, e.g., white blood cells or leukocytes, monocytes, lymphocytes, and platelets). This means that less total colchicine and lower serum levels of colchicine can be therapeutic. Fast accumulating colchicine and high colchicine serum levels, e.g., as for treating an acute gout flare can therefore be avoided, thereby avoiding the known or notorious negative or toxic side effects of colchicine. Thus, lower levels of colchicine, e.g., about 0.1 mg to about 0.75 mg sustained release formulations as described above (or even less frequent doses), are more likely to be sufficient to achieve the desired clinical outcome.

In the normal situation, most colchicine is absorbed primarily in the small intestine and most passes the liver (some is also excreted in the urine via kidney). In the kidney, colchicine is metabolized, but quite a large proportion of colchicine goes through the liver un-metabolized. This means that colchicine travels through the liver into the bile and from there it is excreted into the big intestine (colon). There, colchicine can be resorbed into the body again, which leads to the characteristic second peak (accounts for about 50% of totally absorbed colchicine and is thought to be responsible for gastrointestinal problems, such as diarrhea). If colchicine is formulated as a sustained release preparation as described above, a slower release of colchicine results in a slower resorption. This results in more complete metabolism of colchicine in the liver (because it is less busy with colchicine at a time) and, thus, less recirculation of un-metabolized colchicine. This consequently reduces the incidence of gastrointestinal problems and increases compliance. Colchicine administered as sustained release in accordance with the present invention may also be beneficial for other known side/adverse effects associated with colchicine treatment/administration (the skilled person is well aware of the adverse effects that may occur upon colchicine administration or colchicine treatment). Thus, administration of colchicine as sustained release, as described above, results in a safety increase and safety benefit.

### IV. Methods of Preparing Sustained Release Formulations

The present disclosure envisions various methods of preparing formulations of colchicine, comprising a sustained release component, and an optional immediate release component, wherein colchicine is released from the formulation at a sustained rate that conforms to a pre-determined or desired release profile.

In one embodiment, the colchicine compositions described herein are in the form of a tablet. As used herein, the term “tablet” means a compressed pharmaceutical dosage form of any shape or size. The tablets described herein are, for example, are obtained from the compositions comprising colchicine and a pharmaceutically acceptable excipient. In one embodiment, the colchicine compositions described herein are the form of any dosage form known in the art, specifically, for instance an oral dosage form, for example, a capsule.

In one embodiment, there is provided a controlled release formulation for use in oral dosage forms. The formulation includes a mixture containing hypromellose as a hydrophilic matrix, which is effective to provide controlled release of a pharmaceutically active ingredient such as colchicine.

Matrix systems are well known in the art. In a typical matrix system, the drug is homogenously dispersed in a polymer in association with conventional excipients. This admixture is typically compressed under pressure to produce a tablet. The API (colchicine) is thereby released from the tablet by diffusion and erosion. Matrix systems are described in detail by: (i) Handbook of Pharmaceutical Controlled Release Technology, Ed. D. L. Wise, Marcel Dekker, Inc. New York, N.Y. (2000), and (ii) Treatise on Controlled Drug Delivery, Fundamentals, Optimization, Applications, Ed. A. Kydonieus, Marcel Dekker, Inc. New York, N.Y. (1992), the contents of both of which are hereby incorporated by reference for all purposes.

When the tablet is exposed to aqueous media, such as in the gastrointestinal tract, the tablet surface wets, and the polymer begins to partially hydrate forming an outer gel layer. This outer gel layer becomes fully hydrated and begins to erode into the aqueous fluids. Water continues to permeate toward the core of the tablet permitting another gel layer to form beneath the dissolving outer gel layer. These successive concentric gel layers sustain uniform release of the API by diffusion from the gel layer and exposure through tablet erosion. In the case of the mixtures of the present invention, when included in a compressed tablet matrix, the hypromellose provides a hydrophilic swellable structure capable of functioning as the gel layer. In this way, the drug release is controlled.

In accordance with one embodiment, the colchicine formulation is manufactured by either wet or dry granulation of a colchicine composition, blending the resulting granulate with excipients, and then compressing the composition into tablets.

In one embodiment, wet granulation is used to prepare wet granules comprising colchicine. A granulating liquid is used in wet granulation process. Both aqueous and non-aqueous liquids may be used as the granulating liquid. In one embodiment, the granulating liquid is an aqueous liquid, or more specifically, purified or de-ionized water. The amount of the granulating liquid used depends on many factors, for example, the type of the granulating liquid, the amount of the granulating liquid used, the type of excipient used, the nature of the active agent, and the active agent loading.

In one embodiment, the colchicine particles and suitable excipients are mixed with the granulating liquid for a sufficiently long period to facilitate good distribution of all starting materials and good content uniformity. Wet granulation is generally performed at temperatures between about 20 ° C to about 35 ° C, or more specifically, at room temperature (about 25 ° C). Following wet granulation, the granulate is dried at increased temperatures to yield a dry granulate. In an embodiment, the step of drying is performed for a sufficiently long period until the desired residual moisture content is reached. In an embodiment, this is about 45 ° C for about 12 hours to about 48 hours. It should be appreciated that the overall time to perform the granulation process depends on a variety of factors, including but not limited to, the solvents used, batch size, instruments used, etc.

Any equipment may be used to contact the granulating liquid with the colchicine and the excipients as long as uniform distribution of the granulating liquid is achieved. For example, small-scale production is achieved by mixing and wetting the colchicine and the excipients in mortars or stainless-steel bowls, while for larger quantities, V-blenders with intensifier bars, planetary mixers, rotary granulators, high shear granulators, and fluid-bed granulation equipment may be used. In one embodiment, the granulator is a high shear granulator.

In one embodiment, a method of making a colchicine composition comprises wet granulating colchicine with pharmaceutically acceptable excipients and a granulating liquid to obtain wet granules, and mixing the granules in a next step with a second excipient to obtain a colchicine composition. In one embodiment, the pharmaceutically acceptable excipient comprises a binder and a filler. In an embodiment, the binder is hypromellose. In an embodiment, the filler is lactose monohydrate and pregelatinized starch. In another embodiment, purified water is incorporated as the granulating liquid. In an embodiment, the second excipient mixed with the granules is a filler. In an embodiment, the filler is lactose monohydrate. The colchicine compositions contain, in certain embodiments, about 0.1 wt % to about 10 wt %, or more specifically, about 0.25 wt % to about 0.75 wt %, of colchicine, based on the total weight of the colchicine composition.

In an embodiment, the method of making a composition comprises wet granulating colchicine with a pharmaceutically acceptable excipient to obtain wet granules and mixing the granules with a filler to obtain a colchicine composition. In some embodiments, the method further includes drying the mixture. In another embodiment, the wet granules are dried to obtain dried granules, and then the dried granules are mixed with a binder, a filler, or both to obtain the composition. In another embodiment, the dried granules can be milled to obtain milled granules before mixing the milled dried granules with the binder, a filler, or both. The method further includes, in certain embodiments, mixing the colchicine composition with a glidant, a lubricant, or both to obtain a blend or compressing the blend to obtain a tablet. In one embodiment, the glidant is talc. In another embodiment, the lubricant is stearic acid. The method, in some embodiments, further includes coating the tablet.

In another embodiment, a method of making a colchicine tablet comprises wet granulating colchicine with a pharmaceutically acceptable excipient to obtain wet granules; drying the wet granules to obtain dried granules; milling the dried granules to obtain milled granules; mixing the milled granules with a filler to obtain the composition; mixing the composition with a glidant, a lubricant, or both to obtain a blend; and compressing the blend to obtain a colchicine tablet.

In some embodiments, the wet granules are dried to obtain dried granules before mixing with a second excipient, for example a filler. Wet granules are dried by any suitable means to remove the granulating liquid and to form dried granules containing colchicine and the pharmaceutically acceptable excipient. The conditions and duration of drying depend on factors such as the liquid used and the weight of the granulating particles. Examples of suitable drying methods include, but are not limited to, tray drying, forced air drying, microwave drying, vacuum drying, and fluid bed drying.

After drying, dried granules are able to be mixed directly with an excipient, for example, a filler, a binder, or a lubricant, for further processing. Alternatively, dried granules are optionally subjected to additional processing steps prior to mixing with the excipient. For example, dried granules are, in some embodiments, sized to reduce particle size prior to mixing with an excipient. Exemplary sizing operations include milling or sieving. Any suitable known equipment for reducing the particle size is able to be employed for this purpose.

Suitable excipients are, in some embodiments, added extragranularly and mixed with the granules to form colchicine compositions. As used herein, the term “extragranular” or “extragranularly” means that the referenced material, for example, a suitable excipient, is added or has been added as a dry component after wet granulation. In one embodiment, a filler, a binder, a glidant, and a lubricant are added extragranularly to the granules and mixed to form a blend. In certain embodiments, the blend is encapsulated directly into capsule shells, for example, hard gelatin shells, to form capsule formulations. Alternatively, the blend is compressed into tablets. In some embodiments, the granules are dried granules or milled, dried granules.

Mixing is typically performed for a sufficient time to produce homogeneous mixtures or blends. Mixing is accomplished by blending, stiffing, shaking, tumbling, rolling, or by any other method to achieve a homogeneous blend. In some embodiments, the components to be mixed are combined under low shear conditions in a suitable apparatus, such as a V-blender, tote blender, double cone blender or any other apparatus capable of functioning under low shear conditions.

The homogenous mixtures or blends are then compressed using any method suitable in the industry. The mechanical force will define the physical properties of the tablets, especially the crushing strength of the resulting tablet. The mechanical strength interacts with the initial swelling of the tablet and dilution speed of the tablet core. This effect is well known in the art and can be adjusted and controlled during the lifecycle of the product. For the colchicine sustained-release formulation of the present invention, the compression strengths range from about 30N to about 130N. In one embodiment, the compression strength is about 100N. In another embodiment, the compression strength is about 100N+/−15N.

The colchicine tablets prepared from the above-described methods exhibit acceptable physical characteristics including good friability and hardness. As per EP and USP guidelines, the colchicine tablets disclosed herein have friability in the range of about 0% to less than about 1%.

The colchicine tablet is coated in some embodiments. Coating the tablet is performed by any known process. A coating for the colchicine tablet disclosed herein is any suitable coating, such as, for example, a functional or a non-functional coating, or multiple functional or non-functional coatings. By “functional coating” is meant to include a coating that modifies the release properties of the total formulation, for example, a sustained-release coating. By “non-functional coating” is meant to include a coating that is not a functional coating, for example, a cosmetic coating. A non-functional coating can have some impact on the release of the active agent due to the initial dissolution, hydration, perforation of the coating, etc., but would not be considered to be a significant deviation from the non-coated composition.

In one embodiment, a colchicine composition comprises colchicine, a binder, a filler, a retarding agent, a glidant, and a lubricant. In an embodiment, a colchicine composition comprises about 0.25 to about 0.75 mg colchicine; about 10 to about 80 mg lactose monohydrate; about 5 to about 50 mg pregelatinized starch; about 1 to about 30 mg hypromellose 6 mPa*s; about 5 to about 40 mg RETALAC® (compound of equal parts lactose monohydrate and hypromellose 4000 mPa*s, 50/50 w/w %); about 0.5 to about 5 mg talc; and about 0.5 to about 5 mg stearic acid 50. In an embodiment, the colchicine composition comprises about 0.5 mg colchicine, about 59 mg lactose monohydrate; about 7.5 mg pregelatinized starch; about 1 mg hypromellose 6 mPa*s; about 30 mg RETALAC®; about 1 mg talc; and about 1 mg stearic acid 50. In one embodiment, a described colchicine dosage form has a total weight of about 100 mg. The colchicine composition is, in certain embodiments, formulated in the form of a tablet.

### V. Treatment Methods Using Colchicine to Treat and/or Preventing Chronic Inflammation

Described are methods for treating and/or preventing and/or reducing the occurrence of chronic inflammation in a subject in need thereof, comprising administering a therapeutically effective amount of a colchicine formulation. Colchicine exerts its anti-inflammatory effects primarily by inhibiting microtubule polymerization, which in turn suppresses the activation of the NLRP3 inflammasome, a key component in the inflammatory response. This mechanism is particularly relevant in conditions where excessive inflammation is driven by innate immune responses, such as those observed in certain individuals following COVID-19 vaccination and infection. By modulating these inflammatory pathways, colchicine can alleviate persistent inflammatory symptoms and reduce the risk of associated complications.

In one embodiment, the method involves administering colchicine to a subject exhibiting signs of chronic inflammation post-COVID-19 vaccination, such as elevated levels of pro-inflammatory cytokines (e.g., IL-1β, IL-6) or clinical symptoms like persistent fatigue and myalgia. The treatment aims to restore immune homeostasis by dampening the overactive inflammatory response, thereby improving patient outcomes.

As used herein, “post-vaccination inflammatory syndrome” (PVS) refers to a condition characterized by prolonged inflammatory symptoms following immunization, without evidence of active infection. This may include manifestations such as myocarditis, pericarditis, or systemic inflammatory responses. The methods and formulations described herein address these conditions by targeting the underlying inflammatory mechanisms with colchicine therapy, i.e., administration of a pharmaceutically effective dosage of colchicine or salts thereof to a subject in need thereof.

In a specific embodiment, the method is applied to subjects diagnosed with myocarditis or pericarditis following COVID-19 vaccination. Colchicine's ability to inhibit leukocyte activation and cytokine release makes it a suitable therapeutic agent for reducing cardiac inflammation and preventing further tissue damage in these patients.

COVID-19 infection and, in some cases, COVID-19 vaccination, have been associated with persistent elevation of high-sensitivity C-reactive protein (hs-CRP), a marker of systemic inflammation and vascular stress. Elevated hs-CRP reflects an ongoing low-grade inflammatory response that may contribute to fatigue, endothelial dysfunction, and cardiovascular risk. Colchicine, through its upstream inhibition of pro-inflammatory signaling pathways, has been shown to reduce hs-CRP levels in various inflammatory conditions. This effect indicates that colchicine should be beneficial in managing post-COVID infection or post-vaccination syndromes characterized by sustained hs-CRP elevation.

That is, in another embodiment, the method involves administering colchicine to a subject exhibiting signs of chronic inflammation post-COVID-19 infection, such as elevated levels of pro-inflammatory cytokines (e.g., IL-1β, IL-6, CRP, TNFα, serum amyloid A, and neopterin) or clinical symptoms like persistent fatigue, brain fog, post-exertion malaise (PEM), postural orthostatic tachycardia syndrome (POTS), cough, chest pain, aberrant heart rhythm, joint and muscle pain, rash, sleep problems, and myalgia. More than 200 different Long COVID symptoms have been reported and identified. The methods described herein aim to restore immune homeostasis by dampening the overactive inflammatory response, thereby improving patient outcomes and avoid long term complications of Long COVID such as diabetes, heart conditions, blood clots, and neurological conditions. Brain fog, confusion, and forgetfulness symptoms arising from these conditions are believed to be spurred on by inflammatory signaling pathways and arise from the impact of these signaling pathways on blood vessels in the central nervous system as well as small vessels leading to and surrounding the heart.

As used herein, “Long COVID” refers to a chronic condition characterized by often debilitating inflammatory symptoms following infection with COVID, which emerges typically three or months post-SARS-COV-2-infection. The condition is associated with a wide range of new or ongoing clinical presentations including extreme fatigue, brain fog, and cardiovascular issues such as myocarditis, pericarditis, arrhythmias, heart failure (myocardial infarctions, such as type 2 myocardial infarction), inflammation of the aorta or pulmonary arteries, blood clots, as well as systemic inflammatory responses. Long COVID is believed to be attributed to the presence of viral reservoirs remaining in the subject after infection, autoimmunity mechanisms, and organ damage, to name a few. The methods and formulations described herein address these conditions by targeting the underlying inflammatory mechanisms with colchicine therapy, i.e., administration of a pharmaceutically effective dosage of colchicine or salts thereof to a subject in need thereof.

COVID-19 infection and vaccination also is known to also in some cases lead to an exaggerated immune response characterized by the activation of the NLRP3 inflammasome, a key component of the innate immune system. This activation results in the cleavage of pro-caspase-1 and subsequent release of pro-inflammatory cytokines such as interleukin-1β (IL-1β) and interleukin-18 (IL-18), contributing to systemic inflammation and potential cardiovascular or multisystem complications. IL-1β, in particular, plays a central role in both acute cytokine storms and the chronic inflammatory manifestations observed in some individuals following severe acute respiratory syndrome coronavirus 2 (SARS-COV-2) infection or vaccination. Colchicine exerts its therapeutic effects by disrupting microtubule dynamics, thereby inhibiting the assembly and activation of the NLRP3 inflammasome. This prevents downstream cytokine production, including IL-1B and IL-18, and helps restore immune homeostasis. Accordingly, colchicine formulations described herein are useful for the treatment and/or prevention of chronic inflammatory conditions in which the NLRP3-IL-1B axis plays a pivotal role. By modulating this pathway, colchicine may mitigate inflammation and its associated symptoms in patients with COVID-19-related or vaccine-associated immune dysregulation.

Interleukin-6 (IL-6) is an inflammatory cytokine that is commonly elevated in both acute COVID-19 and in some individuals following vaccination. Persistent IL-6 elevation has been linked to prolonged systemic symptoms and increased cardiovascular risk. By inhibiting upstream mediators such as IL-1ß and reducing neutrophil activation, colchicine indirectly lowers IL-6 production and signaling. This action positions colchicine as a promising treatment for mitigating IL-6-associated inflammation in post-infectious or post-vaccination inflammatory syndromes.

Tumor necrosis factor-alpha (TNF-α) plays a central role in both acute and chronic inflammation and is often elevated in patients with severe COVID-19 or ongoing inflammatory symptoms following immunization. Sustained TNF-α activity contributes to immune dysregulation, tissue injury, and vascular complications. Colchicine, by limiting neutrophil recruitment and cytokine amplification, may suppress TNF-α signaling pathways. This pharmacologic effect provides a rationale for colchicine use in inflammatory states characterized by elevated TNF-α, including those associated with COVID-19 or vaccination.

Myeloperoxidase (MPO), an oxidative enzyme released by activated neutrophils, has been identified as a marker of neutrophil-driven inflammation and oxidative stress in COVID-19 infection and, in some cases, after vaccination. Elevated MPO levels contribute to endothelial injury and have been associated with increased cardiovascular risk. Colchicine's ability to inhibit neutrophil activation and degranulation results in a reduction of MPO release. Accordingly, colchicine may reduce oxidative tissue damage and vascular inflammation in subjects with COVID-19-related or post-vaccination inflammatory syndromes.

Although the presently described methods and formulations are focused on treating chronic inflammation, colchicine is also contemplated herein as being beneficial in the treatment of other inflammatory disorders. In certain embodiments, these include gout, familial Mediterranean fever, Behçet's disease, age-related macular degeneration, and neuroinflammatory conditions such as Alzheimer's disease, all of which involve dysregulated inflammatory pathways. The anti-inflammatory mechanism responsible for chronic inflammation may be applicable to these disease states as well.

Colchicine, according to certain embodiments described herein, is used in combination with conventional therapy for any of the diseases disclosed herein. Such conventional therapies are well known in the art and the skilled person knows any such therapies. Colchicine as described herein may also be used in combination with colchicine-compatible statins. In general, non-limiting examples of statins are atorvastatin (LIPITOR®, TORVAST®), fluvastatin (LESCOL®), lovastatin (MEVACOR®, ALTOCOR®, ALTOPREV®), pitavastatin (LIVALO®, PITAVAR), pravastatin (PRAVACHOL®, SELEKTINE®, LIPOSTAT®), rosuvastatin (CRESTOR®) and simvastatin (ZOCOR®, LIPEX®). In connection with the present disclosure, colchicine-compatible statins are also contemplated as being combined with the colchicine formulations described herein. Preferably, in the context of the presently described methods, the combination of the compositions described herein with the colchicine-compatible statins are accomplished in connection with stable coronary heart disease. Such statins are statins which, for example, due to the nature of their mechanism of action, metabolism and/or clearance do not, or only to a small extent, interfere with the mechanism of action, metabolism and/or clearance of colchicine and therefore show a reduced risk, severity and/or incidence of drug-related drug adverse events when given in combination with colchicine.

In an embodiment, the use of colchicine is in fixed (within the same pharmaceutical preparation) or unfixed (different pharmaceutical preparation) combinations with the above-mentioned additional active pharmaceutical agents. “Fixed combination” is to be understood as meaning a combination whose active ingredients are combined at fixed doses in the same vehicle (single formula) that delivers them together to the point of application. Fixed combination can mean, e.g., in a single tablet, solution, cream, capsule, gel, ointment, salve, patch, suppository or transdermal delivery system. “Unfixed combination” as used herein is to be understood as meaning that the active ingredients/components are in more than one vehicle (e.g. tablets, solutions, creams, capsules, gels, ointments, salves, patches, suppositories or transdermal delivery systems). Each of the vehicles can contain a desired pharmaceutical composition or active component. For example, a preferred unfixed combination as described herein means that one vehicle contains colchicine, as described herein, and another vehicle contains a colchicine-compatible statin, as described herein. Examples of colchicine as described herein in fixed or unfixed combination(s) encompass(es) colchicine in combination with one or more colchicine-compatible statins selected from the group consisting of atorvastatin, rosuvastatin, simvastatin and pravastatin. Specifically, colchicine as described herein in fixed or unfixed combination is to be understood as meaning colchicine in combination with atorvastatin. Specifically, colchicine as described herein in fixed or unfixed combination is to be understood as meaning colchicine in combination with rosuvastatin. Specifically, colchicine as described herein in fixed or unfixed combination is to be understood as meaning colchicine in combination with simvastatin. Specifically, colchicine as described herein in fixed or unfixed combination is to be understood as meaning colchicine in combination with pravastatin.

In some embodiments, colchicine as described herein may be used in combination with a statin and another agent, such as ezetimibe/simvastatin. Colchicine as described herein may be used in combination with other drugs, e.g., which are used in medicine and are known to the skilled person (e.g. antibiotics, NSAID (non-steroidal anti-inflammatory drugs), corticosteroids).

### VI. Administration Methods

Methods of administering the colchicine formulations described herein to a subject in need thereof are well known to or are readily determined by those skilled in the art. The route of administration of the colchicine formulation is, for example, oral, parenteral, by inhalation, or topical. The term parenteral as used herein includes, e.g., intravenous, intraarterial, intraperitoneal, intramuscular, subcutaneous, rectal, or vaginal administration.

Colchicine formulations described herein include, in some embodiments, one or more pharmaceutically acceptable carriers and thus are prepared in the form of a local formulation, in order for it to be administered. The pharmaceutically acceptable carrier is, for example, saline, sterile water, linger liquid, buffer saline, a dextrose solution, a malto dextrin solution, glycerol, ethanol and mixtures of one or more thereof, and also may include an additive such as an antioxidant, a buffer, a bacteriostatic agent or the like, as necessary. Furthermore, in some embodiments, a diluent, a dispersant, a surfactant, a binder, and a lubricant is added when the compositions described herein are prepared, e.g., in the form of a local formulation such as an ointment, lotion, cream, gel, skin emulsion, skin suspension, patch, or spray.

Non-limiting examples for administration of the compound and or compositions described herein include, for example, coated and uncoated tablets, soft gelatine capsules, hard gelatine capsules, lozenges, troches, solutions, emulsions, suspensions, syrups, elixers, powders and granules for reconstitution, dispersible powders and granules, medicated gums, chewing tablets, and effervescent tablets. The composition described herein are administered in any pharmaceutical form for oral (e.g. solid, semi-solid, liquid), dermal (e.g. dermal patch), sublingual, parenteral (e.g. injection), ophthalmic (e.g. eye drops, gel or ointment), or rectal (e.g. suppository) administration. In an embodiment, the composition is formulated as a tablet, capsule, suppository, dermal patch, or sublingual formulation.

The pharmaceutical compositions described herein comprise, in some embodiments, one or more pharmaceutically acceptable carriers, including, e.g., ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol, and wool fat.

Certain pharmaceutical compositions described herein are orally administered in an acceptable dosage form including, e.g., capsules, tablets, aqueous suspensions, or solutions. Certain pharmaceutical compositions described herein are administered by nasal aerosol or inhalation. Such compositions are prepared as solutions in saline, employing benzyl alcohol or other suitable preservatives, absorption promoters to enhance bioavailability, and/or other conventional solubilizing or dispersing agents.

The amount of the colchicine formulation to be combined with the carrier materials to produce a single dosage form will vary depending upon the host/patient/subject being treated and the particular mode of administration thereto. The composition is administered in some embodiments as a single dose, multiple doses, or over an established period of time in an infusion. Dosage regimens also can be adjusted to provide the optimum desired response (e.g., a therapeutic or prophylactic response).

In some embodiments, the compositions described herein are parenterally administered. Preparations for parenteral administration include, for example, sterile aqueous or non-aqueous solutions, suspensions, and emulsions. Examples of non-aqueous solvents include propylene glycol, polyethylene glycol (PEG), vegetable oils such as olive oil, and injectable organic esters such as ethyl oleate. Aqueous carriers include, e.g., water, alcoholic/aqueous solutions, emulsions or suspensions, including saline and buffered media. In some embodiments, pharmaceutically acceptable carriers include, but are not limited to, 0.01-0.1 M, or 0.05 M, phosphate buffer or 0.8% saline. Other common parenteral vehicles include, for example, sodium phosphate solutions, Ringer's dextrose, dextrose and sodium chloride, lactated Ringer's, or fixed oils. Intravenous vehicles include, for example, fluid and nutrient replenishers, electrolyte replenishers, such as those based on Ringer's dextrose, and the like. Preservatives and other additives are also included in the described compositions in certain embodiments, such as, for example, antimicrobials, antioxidants, chelating agents, inert gases, and the like.

Parenteral formulations can be a single bolus dose, an infusion, or a loading bolus dose followed with a maintenance dose. These compositions are, for example, administered at specific fixed or variable intervals, e.g., once a day, or on an “as needed” basis.

The composition described herein are, in some embodiments, administered in a dose range varying depending on the patient's body weight, age, gender, health condition, diet, administration time, administration method, excretion rate, and disease severity. The compounds described herein are compounds per se in their use as pharmacophores or as pharmaceutical compositions and are administered to the patient and/or subject at a suitable dose. The dosage regiment is determined by the attending physician based on the identity of the subject and other commonly consulted clinical factors. As is well known in the medical arts, dosages for any one patient depends upon many factors, including the patient's size, body surface area, age, the particular compound to be administered, sex, time, and route of administration, general health, and other drugs being concurrently administered. Generally, the regimen as a regular administration of the pharmaceutical composition comprising herein defined should be, e.g., in a range as described below. Progress is able to be monitored by periodic assessment, such as, for example, by blood test, testing for the aforementioned inflammation factors, for example.

Periodic assessment, in some embodiments, is by way of one or more blood-based inflammatory biomarker assays known in the art, for instance blood-based assays for detecting the relative level of one or more inflammatory markers such as CRP or hs-CRP, IL-1 or IL-1β, IL-6, TNF-α, and MPO, Such blood-based assays also include, but are not limited to, erythrocyte sedimentation rate (ESR), plasma viscosity, serum ferritin, white blood cell (WBC) count, and cytokine-specific assays, e.g., assays detecting the level of inflammatory biomarkers as described herein. Typical human levels of such inflammatory biomarkers include the following: CRP—about 3 mg/L or less, ESR—about 20 mm/hr or less, ferritin—about 20 to about 200 mcg/L, IL-6—less than about 10 μg/mL, TNFα—mean level of about 2.23 μg/mL to about 5.5 μg/mL, IL-1—below about 1 μg/mL, interferon (IFN) γ—about 13 μg/mL or less, IL-10—about 1.8 to about 14.7 μg/mL. Typical blood detection assays for cytokines include commercially available enzyme-linked immunosorbent (ELISA) assays, multiplex immunoassays (Luminex, etc.), cytokine antibody arrays (membrane or bead-based), proximity-based assays (ProQuantum from Invitrogen, etc.), and no-wash/functional assays (LUMIT® from Promega, etc.). In some embodiments, a blood sample from the subject is assayed, in others, one or more of serum samples, plasma samples, tissue culture supernatants, and/or saliva samples are assayed. In some instances, the progress of treatment is followed by periodic full cytokine blood tests. In some embodiments, the protein level, or protein breakdown product, of the cytokine is detected in the sample, in others, mRNA expression is detected. In some embodiments, cytokine detection is by way of both protein and mRNA.

The compositions described herein are administered with a single dose or with 2, 3, 4, 5, 6, 7, 8, 9, or 10 doses, if desired. The composition are administered, for example, 1, 2, 3, 4, 5, 6, 7, 8, 9, or 10 times per day. In some embodiments, colchicine compositions described herein are administered once per day. In other embodiments, colchicine compositions described herein are administered once per day as a single dose.

The compositions described herein are, for example, in some embodiments, administered regularly for long periods of time. In an embodiment, the composition is administered regularly for 1, 2, 3, 4, 5, 6, 7, 8, 9, 10, or more years. In another embodiment, the composition is administered regularly for 1, 2, 3, 4, 5, 6, 7, 8, 9, 10, 11, 12, or more months. In other embodiments, the composition is administered regularly for 1, 2, 3, 4, 5, 6, 7, 8, 9, 10, or more weeks. As used herein, the term “regularly” refers to administration of the composition at regular times or intervals over a period of time. For instance, the composition is in some embodiments administered to a patient once daily for three years. In other embodiments, the composition is administered to a patient once every other day for 5 years. It should be appreciated that the frequency of administration is variable based on a number of factors, including, but not limited to, the severity of disease, the overall health of the patient, any additional medications the patient is taking, and whether the treatment is prophylactic or not. It should also be appreciated that the frequency of administration is adjustable at any point.

The amount/concentration/dose of the compositions described herein are, for example, between 0.1 mg and 5.0 mg, 0.1 mg and 2.0 mg, 0.1 mg to 1.5 mg, 0.1 mg to 1.0 mg, 0.1 mg to 0.75 mg, 0.1 mg to 0.5 mg, 0.25 mg to 5.0 mg, 0.25 mg to 2.0 mg, 0.25 mg to 1.5 mg, 0.25 mg to 1.0 mg, 0.25 mg to 0.75 mg or 0.25 mg to 0.5 mg. In an embodiment, the composition is administered at a daily dose of colchicine of between about 0.1 mg and about 0.75 mg or between about 0.1 mg and about 0.5 mg. In another embodiment, the composition is administered at a daily dose of colchicine of between about 0.25 mg to about 0.75 mg or between about 0.25 mg to about 0.5 mg. In an embodiment, the composition is administered at a daily dose of about 0.5 mg colchicine.

In an embodiment, the amount/concentration of colchicine as used herein is administered at the first day of administration in a higher dose (concentration/amount) compared to the administration of colchicine at the following days(s) of administration (maintenance administration/maintenance dose of administration). Alternatively, such decreased dose (maintenance dose) is initiated after 2, 3, 4, 5, 6, 7, 8, 9, or 10 days of initial administration of the higher dose. In case the course of treatment is any such as described above, the higher dose/amount/concentration of colchicine (e.g. at the first day of administration) is any as described above, provided that the maintenance dose (the dose/amount/concentration of colchicine at the days following the higher dose/amount/concentration) is lower than the initial dose/amount/concentration of colchicine (e.g. at the first day of administration). In one embodiment, the composition is administered with a dose of colchicine of between about 1.0 mg to about 2.0 mg at the first day (for instance, as a single dose) of administration and the maintenance dose of colchicine at the following day(s) of administration is between about 0.5 mg to about 1.0 mg.

In keeping with the scope of the present disclosure, in one embodiment, the colchicine formulation of described herein is administered to a human or other animal in accordance with the aforementioned methods of treatment in an amount sufficient to produce a therapeutic effect. The colchicine formulation is administered to such human or other animal in a conventional dosage form prepared by combining the colchicine formulations described herein optionally with a conventional pharmaceutically acceptable carrier or diluent according to known techniques. It will be recognized by one of skill in the art that the form and character of the pharmaceutically acceptable carrier or diluent is dictated by the amount of active ingredient with which it is to be combined, the route of administration and other well-known variables.

By “therapeutically effective dose or amount” or “effective amount” is intended an amount of the colchicine formulation that when administered brings about a positive therapeutic response with respect to treatment of a patient with a disease to be treated, e.g., an improvement in the disease can be evidenced by, for example, a delayed onset of clinical symptoms of the disease or condition, a reduction in severity of some or all clinical symptoms of the disease or condition, a slower progression of the disease or condition, an improvement in the overall health or well-being of the subject, or by other parameters well known in the art that are specific to the particular disease.

Also contemplated herein are embodiments encompassing the use of the colchicine formulation in the manufacture of a medicament for treating a subject for treating a cardiovascular disease and/or inflammatory disorder, wherein the medicament is used in a subject that has been pretreated or is concurrently being treated with at least one other therapy. By “pretreated” or “pretreatment” is intended the subject has received one or more other therapies prior to receiving the medicament comprising the colchicine formulation. “Pretreated” or “pretreatment” includes subjects that have been treated with at least one other therapy within 2 years, within 18 months, within 1 year, within 6 months, within 2 months, within 6 weeks, within 1 month, within 4 weeks, within 3 weeks, within 2 weeks, within 1 week, within 6 days, within 5 days, within 4 days, within 3 days, within 2 days, or even within 1 day prior to initiation of treatment with the medicament comprising the colchicine formulation. By “concurrent” or “concomitant” is intended the subject is receiving one or more other therapies while at the same time receiving the medicament comprising the colchicine formulations described herein. It is not necessary that the subject was a responder to pretreatment with the prior therapy or therapies or a responder to the concurrent therapy or therapies. Thus, the subject that receives the medicament comprising the colchicine formulation could have responded, or could have failed to respond, to pretreatment with the prior therapy, or to one or more of the prior therapies where pretreatment comprised multiple therapies.

All of the references cited above, as well as all references cited herein, are incorporated herein by reference in their entireties.

While the described methods and compositions have been illustrated and described in detail in above, such illustration and description are to be considered illustrative or exemplary and not restrictive. It will be understood that changes and modifications are possible as understood by those of ordinary skill within the scope and spirit of the following claims. In particular, the described methods and formulations are contemplated as encompassing further embodiments with any combination of features from different embodiments described above and below.

The presently disclosed methods and formulations are additionally described by way of the following illustrative non-limiting examples that provide a better understanding of their many advantages. The following examples are included to demonstrate certain embodiments. It should be appreciated by those of skill in the art that the techniques disclosed in the examples which follow represent techniques used herein to function well in the practice of the described methods and formulations, and thus are considered to constitute certain contemplated modes for its practice. However, those of skill in the art should, in light of the present disclosure, appreciate that many changes can be made in the specific embodiments which are disclosed and still obtain a like or similar result without departing from the spirit and scope described herein.

### EXAMPLES

### Example 1: Colchicine Sustained Release Tablet

This example illustrates a colchicine sustained release tablet. The tablet uses the ingredients and concentrations shown in Table 1 below

| | mg/ | Tablet | |
|---|---|---|---|
| Ingredient | Tablet | % | Function |
| Colchicine | 0.500 | 0.5 | Active Pharmaceutical |
| | | | Ingredient (API) |
| Lactose monohydrate | 59.00 | 59.0 | Filling Agent |
| Pregelatinized Starch | 7.50 | 7.5 | Filling Agent |
| Hypromellose 6 mPa*s | 1.000 | 1.0 | Binder |
| Purified water¹ | q.s. | q.s. | Diluent for API and Binder |
| Lactose monohydrate | 10.00 | 10.00 | Filling Agent |
| Retalac (Compound of | 20.00 | 20.00 | Retarding Agent |
| Lactose monohydrate | | | |
| and Hypromellose | | | |
| 4000 mPa*s | | | |
| 50/50 w/w %) | | | |
| Talc | 1.00 | 1.0 | Glidant |
| Stearic acid 50 | 1.00 | 1.0 | Lubricant |
| Total tablet weight | 100.00 | | |
| [mg]: | | | |

¹removed within the process

The concentrations may be altered to change certain properties of the formulations, for instance, the dissolution profile. Table 2 shows the ranges for each ingredient.

| Ingredient | Range | Function |
|---|---|---|
| Colchicine | 0.5-0.75 | Active Pharmaceutical |
| | | Ingredient (API) |
| Lactose monohydrate | 10-80 | Filling Agent |
| Pregelatinized Starch | 5-50 | Filling Agent |
| Hypromellose 6 mPa*s | 1-30 | Binder |
| Purified water¹ | q.s. | Diluent for API and Binder |
| Lactose monohydrate | 10-30 | Filling Agent |
| Retalac (Compound of | 5-40 | Retarding Agent |
| Lactose monohydrate | | |
| and Hypromellose | | |
| 4000 mPa*s | | |
| 50/50 w/w %) | | |
| Talc | 0.5-5 | Glidant |
| Stearic acid 50 | 0.5-5 | Lubricant |
| Total tablet weight | | |
| [mg]: | | |

¹removed within the process

### Example 2: Method of Making a Colchicine Sustained Release Tablet

The above ingredients are utilized to make a tablet to the following working directions:

Granulation: The granulation was performed in a Kenwood mixer. The colchicine and hypromellose 6 Mpa*s were first weighed and separately dissolved into purified water. This solution of hypromellose 6 Mpa*s was filled in the mixer containing the lactose within 1.5 min followed by a 3 minute mixing time. Subsequently, the dissolved colchicine was sprayed with a filing agent (e.g., lactose monohydrate) under continuous mixing over a period of 15 minutes. These steps were performed at room temperature. The wet granulate was then passed through a 1.0 mm hand sieve. It was then dried in an oven (Haeraeus) at 45 ° C for 26 h to a residual moisture content of 0.53%, and passed through a 0.8 mm sieve shaker (Erweka AR 400). Density parameters were tested (Engelmann). Bulk density 0.53 g/ml, compacted bulk density 0.67 g/ml, Hausner ratio 1.26. Rheology: Flow time 4 sec; Slope angle 23.8°.

Blending: Following the granulation process, the granulate is compounded with a filling agent (e.g., lactose monohydrate), a retarding agent (e.g., Retalac), and other excipients (e.g., flow enhancer, glidants and/or lubricants) to support the tablet compression process. To this end, these ingredients were placed manually through a 0.8 mm sieve and mixed with the granulate in a cube mixer (Erweka) for 10 minutes. In one embodiment, the glidant used may be Talc. In another embodiment, the lubricants used may be Stearic acid. The granulate will then be blended using a suitable mixer.

Compression of Tablets: To form the tablets, a compression force is needed. The mechanical force will define the physical properties of the tablets, especially the crushing strength of the resulting tablet. The mechanical strength interacts with the initial swelling of the tablet and dilution speed of the tablet core. This effect is well known in the art and can be adjusted and controlled during the lifecycle of the product.

Tableting was performed on a Korsch (EK 0) tablet press with a round tabletting tool, biconvex, 6 mm in diameter. Average tablet hardness was approximately 100N+/−15N. Tablets measured about 100 mg in mass, friability was not measurable. Breaking strength and hardness were measured with a Erweka Multickeck. Friability was measured with a Erweka Friabilator and a Mettler analytical balance. The dimensions were measured using a Mitutoyo caliper.

### Example 3: Measurements of Dissolution Profiles of Sustained-Release Colchicine Formulations

The dissolution of the sustained release formulation of colchicine was measured at various time points. The compositions were dissolved in 500 ml of water at 37 ° C and stirred continuously over a period of 6 hours. Samples were drawn at several time points to study the kinetics of the dissolution process of the drug substance within the hydrophilic matrix system. Colchicine content in the samples was analyzed using HPLC analysis.

Several batches were tested to determine the optimal dissolution profile for the sustained release formulation. The release can be modified by both the concentration of hypromellose or by using different viscosity grades of hypromellose (e.g. 1000 mPa or 10,000 mPa). In the batches tested below, the viscosity grade remained constant, however, the concentration of hypromellose 4000 mPa in the tablet was modified.

Table 3 below summarizes the various compositions that were tested.

| | | Mass per | Per batch | | |
|---|---|---|---|---|---|
| Material name | Mass [%] | Tbl. [mg] | [g] | −0.5% | +0.5% |

Stem granulate: (common for 3 Tablet mixings)

| | | | | | |
|---|---|---|---|---|---|
| PE Colchicine | 0.500 | 0.500 | 7.50 | 7.46 | 7.54 |
| Lactose monohydrate EP | 59.000 | 59.000 | 885.00 | 880.6 | 889.4 |
| Pregelatinized Starch USP | 7.500 | 7.500 | 112.50 | 111.94 | 113.06 |
| FB Hypromellose 6 mPa*s | 1.000 | 1.000 | 15.00 | 14.93 | 15.08 |
| EP/JP/USP | | | | | |
| Water purified * for | 0.000 | 4.000 | 60.00 | 59.70 | 60.30 |
| Colchicine | | | | | |
| Water purified * | 0.000 | 8.300 | 124.50 | 123.88 | 125.1 |
| For Hypromellose | | | | | |

Batch 1: Tableting mix 10% Retalac (compression strength = 100N):

| | | | | | |
|---|---|---|---|---|---|
| Lactose monohydrate EP | 20.000 | 20.000 | 80.0 | 79.6 | 80.4 |
| (Filling agent ad 100 | | | | | |
| mg per tablet) | | | | | |
| PE Retalac (50% Lactose/50% | 10.000 | 10.000 | 40.00 | 39.8 | 40.2 |
| Hypromellose | | | | | |
| 4000 mPas) | | | | | |
| Talc EP/JP | 1.000 | 1.000 | 4.00 | 3.98 | 4.02 |
| Stearic acid 50 EP | 1.000 | 1.000 | 4.00 | 3.98 | 4.02 |
| Total tablet weight: | 100.000 | 100.000 | | | |

Batch 2: Tableting mix 15% Retalac (compression strength = 100N):

| | | | | | |
|---|---|---|---|---|---|
| Lactose monohydrate EP | 15.000 | 15.000 | 60.00 | 59.7 | 60.3 |
| (Filling agent ad 100 | | | | | |
| mg per tablet) | | | | | |
| PE Retalac (50% Lactose/50% | 15.000 | 15.000 | 60.00 | 59.7 | 60.3 |
| Hypromellose | | | | | |
| 4000 mPas) | | | | | |
| Talc EP/JP | 1.000 | 1.000 | 4.00 | 3.98 | 4.02 |
| Stearic acid 50 EP | 1.000 | 1.000 | 4.00 | 3.98 | 4.02 |
| Total tablet weight: | 100.000 | 100.000 | | | |

Batch 3: Tableting mix 20% Retalac (compression strength = 100N):

| | | | | | |
|---|---|---|---|---|---|
| Lactose monohydrate EP | 10.000 | 10.000 | 40.00 | 79.6 | 80.4 |
| (Filling agent ad 100 | | | | | |
| mg per tablet) | | | | | |
| PE Retalac (50% Lactose/50% | 20.000 | 20.000 | 80.00 | 79.6 | 80.4 |
| Hypromellose | | | | | |
| 4000 mPas) | | | | | |
| Talc EP/JP | 1.000 | 1.000 | 4.00 | 3.98 | 4.02 |
| Stearic acid 50 EP | 1.000 | 1.000 | 4.00 | 3.98 | 4.02 |
| Total tablet weight: | 100.000 | 100.000 | | | |

Batch 4: Tableting mix 30% Retalac (compression strength = 50N and 130N):

| | | | | | |
|---|---|---|---|---|---|
| Lactose monohydrate EP | 0.000 | 0.000 | 00.00 | 0.0 | 0.0 |
| (Filling agent ad 100 | | | | | |
| mg per tablet) | | | | | |
| PE Retalac (50% Lactose/50% | 30.000 | 30.000 | 120.00 | 119.40 | 120.60 |
| Hypromellose | | | | | |
| 4000mPas) | | | | | |
| Talc EP/JP | 1.000 | 1.000 | 4.00 | 3.98 | 4.02 |
| Stearic acid 50 EP | 1.000 | 1.000 | 4.00 | 3.98 | 4.02 |
| Total tablet weight: | 100.000 | 100.000 | | | |

Batch 5: Tableting mix 0% Retalac (compression strength = 100N):

| | | | | | |
|---|---|---|---|---|---|
| Lactose monohydrate EP | 30.000 | 30.000 | 120.00 | 119.40 | 120.60 |
| (Filling agent ad 100 | | | | | |
| mg per tablet) | | | | | |
| PE Retalac (50% Lactose/50% | 0.000 | 00.000 | 00.00 | 0.0 | 0.0 |
| Hypromellose | | | | | |
| 4000 mPas) | | | | | |
| Talc EP/JP | 1.000 | 1.000 | 4.00 | 3.98 | 4.02 |
| Stearic acid 50 EP | 1.000 | 1.000 | 4.00 | 3.98 | 4.02 |
| Total tablet weight: | 100.000 | 100.000 | | | |

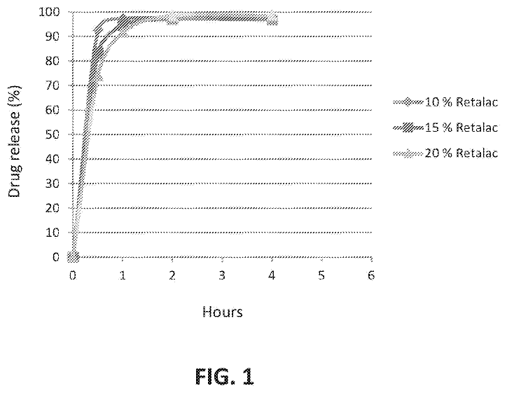

The dissolution profiles of the various batches are provided in Tables 4-6 and FIGS. 1-4. In particular, the dissolution profile for Batches 1-3 is summarized in Table 4 and FIG. 1. The profile for Batch 1 shows approximately a 92% release within about 30 minutes, followed by a constant release. Complete dissolution occurred after 2 hours. Batch 2 shows approximately a 83% release within about 30 minutes, followed by a constant release. Complete dissolution occurred after 2 hours. Batch 3 shows approximately a 74% release within about 30 minutes, followed by a constant release. Complete dissolution occurred after 2 hours.

TABLE 4

| Concentration | | Hour | Hour | Hour | Hour | Hour |
|---|---|---|---|---|---|---|
| Retardant | Sample | 0 | 0.5 | 1 | 2 | 4 |
| 10% Retalac | 1. | | 90.6 | 98.1 | 99.1 | 98.9 |
| | 2. | | 93.7 | 97.6 | 97.5 | 97.6 |
| | 3. | | 98.6 | 99.3 | 99.5 | 99.8 |
| | 4. | | 93.7 | 97.5 | 98.1 | 98.2 |
| | 5. | | 89.9 | 93.7 | 93.7 | 93.8 |
| | 6. | | 89.6 | 97.2 | 97.5 | 97.6 |
| | Mean | 0 | 92.7 | 97.2 | 97.6 | 97.7 |
| 15% Retalac | 1. | | 75.1 | 89.2 | 95.9 | 96.0 |
| | 2. | | 74.5 | 91.3 | 95.0 | 94.9 |
| | 3. | | 85.7 | 99.1 | 100.3 | 100.2 |
| | 4. | | 86.9 | 98.2 | 100.1 | 99.3 |
| | 5. | | 84.7 | 93.9 | 94.6 | 94.3 |
| | 6. | | 90.7 | 96.0 | 95.7 | 96.1 |
| | Mean | 0 | 82.9 | 94.6 | 96.9 | 96.8 |
| 20% Retalac | 1. | | 68.1 | 87.7 | 97.8 | 97.8 |
| | 2. | | 62.0 | 83.2 | 95.2 | 96.3 |
| | 3. | | 85.4 | 95.2 | 98.2 | 98.3 |
| | 4. | | 76.6 | 94.7 | 97.2 | 96.9 |
| | 5. | | 71.8 | 94.5 | 103.5 | 103.1 |
| | 6. | | 80.6 | 95.4 | 99.0 | 98.9 |
| | Mean | 0 | 74.1 | 91.8 | 98.5 | 98.6 |

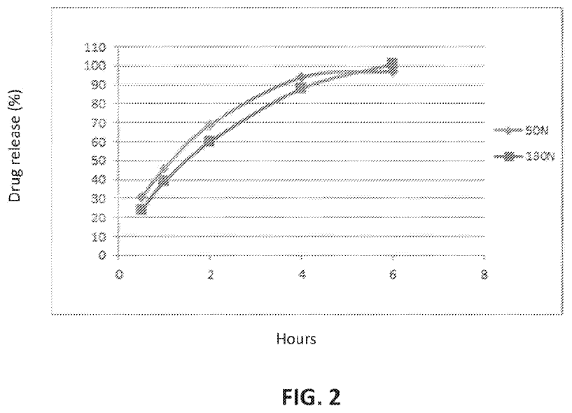

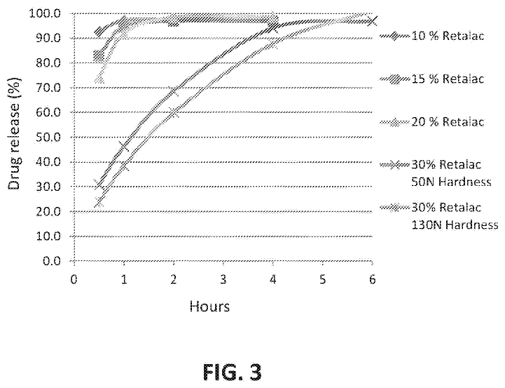

The dissolution profile for Batch 4 is summarized in Table 5 and FIG. 2. Batch 4A shows approximately a 30% release within about 30 minutes, followed by a constant release. Complete dissolution occurred after 6 hours. Batch 4B shows approximately a 23% release within about 30 minutes, followed by a constant release. Complete dissolution occurred within 6 hours. In this example, a difference in tablet hardness results in a difference in release rates. FIG. 3 shows the dissolution profiles for Batches 1-4.

TABLE 5

| Concentration | | Hour | Hour | Hour | Hour | Hour |
|---|---|---|---|---|---|---|
| Retardant | Sample | 0.5 | 1 | 2 | 4 | 6 |
| (A) | No.1 | 30.5 | 47.1 | 70.7 | 96.5 | 96.9 |
| 30% Retalac | No.2 | 34.5 | 49.3 | 70.6 | 95 | 98 |
| 50N Hardness | No.3 | 27.8 | 42.6 | 64.6 | 90.9 | 95.8 |
| | | 30.9 | 46.3 | 68.6 | 94.1 | 96.9 |
| (B) | No.4 | 22.5 | 37.1 | 54.9 | 81.2 | 92.5 |
| 30% Retalac | No.5 | 25.9 | 42 | 67.1 | 94.5 | 108.7 |
| 130N Hardness | No.6 | 23.3 | 36.5 | 57.8 | 87.9 | 101.8 |
| | | 23.9 | 38.5 | 59.9 | 87.9 | 101.0 |

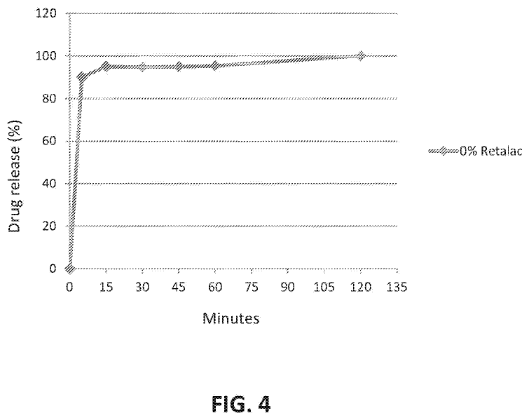

The dissolution profile for Batch 5 is summarized in Table 6 and FIG. 4. Batch 5 represents the immediate release version of the composition. The profile for Batch 5 shows a 90% release within 5 minutes. Complete dissolution occurred within 2 hours.

TABLE 6

| Concentration | | min | min | min | min | min | min | min |
|---|---|---|---|---|---|---|---|---|
| Retardant | Sample | 0 | 5 | 15 | 30 | 45 | 60 | 120 |
| 0% Retalac | 1. | | 92.2 | 96.8 | 96.8 | 96.9 | 97.2 | |
| | 2. | | 96.7 | 100.6 | 99.9 | 100.3 | 100.7 | |
| | 3. | | 94.7 | 100.8 | 100.5 | 100.9 | 101.1 | |
| | 4. | | 86.5 | 92.7 | 92.9 | 93.1 | 93.3 | |
| | 5. | | 85.6 | 91 | 91.7 | 91.4 | 91.8 | |
| | 6. | | 84.5 | 88.1 | 87.1 | 87.1 | 87.5 | |
| | Mean | 0 | 90.0 | 95.0 | 94.8 | 95.0 | 95.0 | 100 |

Table 7 below summarizes another batch, Batch 6, that was tested.

| | | Mass per | Per | | |
|---|---|---|---|---|---|
| | Mass | Tbl. | batch | Range for | Weighting |
| Material name | [%] | [mg] | [g] | variation | Tolerance |
| PE Colchicine | 0.550 | 0.550 | 5.5 | | 0.5% |
| Lactose monohydrate EP | 58.950 | 58.950 | 589.50 | | 1% |
| Pregelatinized Starch | 7.500 | 7.500 | 75.00 | | 1% |
| USP | | | | | |
| FB Hypromellose | 1.000 | 1.000 | 10.00 | | 1% |
| 6 mPa*s EP/JP/USP | | | | | |
| Water purified * for | 0.000 | 4.000 | 40.00 | | 1% |
| Colchicine | | | | | |
| Water purified * | 0.000 | 8.300 | 83.00 | | 1% |
| For Hypromellose | | | | | |

Tableting mix 25% Retalac (compression strength = 55N):

| | | | | | | |
|---|---|---|---|---|---|---|
| Lactose monohydrate EP | 5.00 | 5.00 | 50.00 | 2.5% | 10% | 1% |
| (Filling agent ad 100 mg | | | | | | |
| per tablet) | | | | | | |
| PE Retalac | 25.00 | 25.00 | 250.00 | 20% | 27.5% | 1% |
| (50% Lactose/ | | | | | | |
| 50% Hypromellose | | | | | | |
| 4000 mPas) | | | | | | |
| Talc EP/JP | 1.000 | 1.000 | 10.00 | | | 1% |
| Stearic acid 50 EP | 1.000 | 1.000 | 10.00 | | | 1% |
| Total tablet weight: | 100.000 | 100.000 | 1,000.00 | | | |
| Related Substances | NMT 2.5% | | | | | |
| N-Deacetyl-N-formyl- | NMT 1 % | | | | | |
| Colchicine (imp A) | NMT 3.5% | | | | | |
| unknown impurities | | | | | | |
| Total impurities | | | | | | |

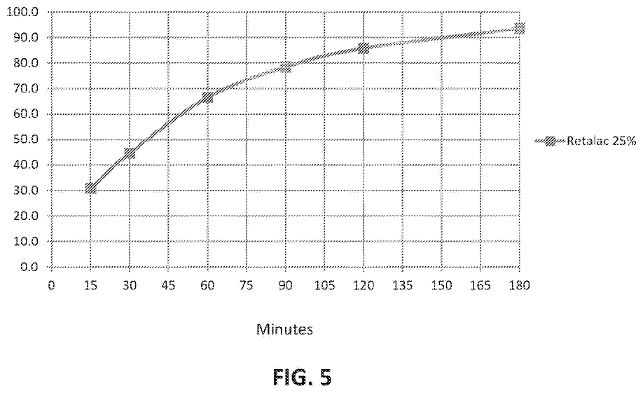

The dissolution profile for Batch 6 is summarized in Table 7 and FIG. 5A. Batch 6 represents a sustained release version of the composition. The profile for Batch 6 in FIG. 5A shows about a 45% release within 30 minutes, about a 65% release in 60 minutes, and about a 80% release in 90 minutes. Complete dissolution occurs within about 2 hours.

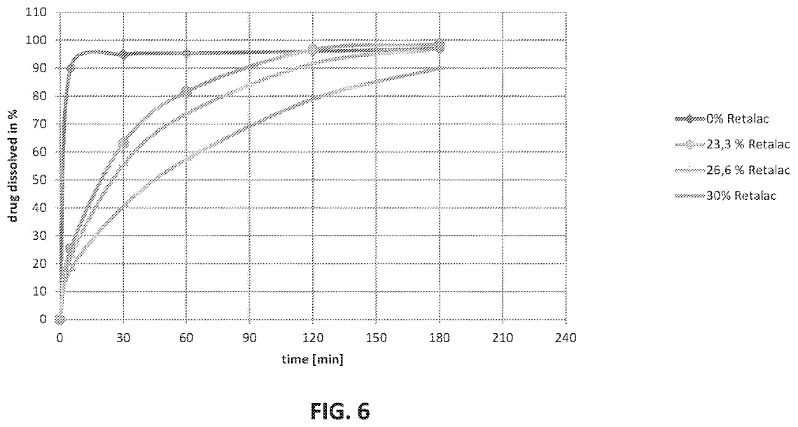

Batch 4 was further modified by altering the concentration of retarding agent (i.e. Retalac) in the composition (Retalac 0%, 23.3%, 26.6% or 30%). The dissolution profiles of the four compositions are shown in FIG. 6. As shown in FIG. 6, the composition with 23.3% Retalac shows about a 65% release within 30 min, about a 80% release in 60 min, and about a 90% release in 90 min. Complete dissolution occurs within about 120 min. As further shown in FIG. 6, the composition with 26.6% Retalac shows about a 55% release within 30 min, about a 75% release in 60 min, and about a 85% release in 90 min. Complete dissolution occurs within about 180 min. As further shown in FIG. 6, the composition with 30% Retalac shows about a 40% release within 30 min, about a 55% release in 60 min, and about a 70% release in 90 min. Complete dissolution occurs in more that about 180 min. As further shown in FIG. 6, the composition with 0% Retalac (i.e., immediate release composition) shows complete dissolution within about 15 min.

As mentioned previously, the release profile of the sustained release composition can be changed to a specific or desired target release by adjusting the amount of retarding agent (i.e., Retalac) as well as tablet hardness. The release depends upon a variety of factors, including erosion of the outer layer of colchicine (i.e., the immediate release portion) as well as diffusion of the inner layer of colchicine (i.e., the sustained release portion). Since the percentage of colchicine is low in the sustained release formulation and the tablets are small, this balance between erosion and diffusion is very sensitive and has to be fine-tuned to reach a very specific dissolution profile.

### Example 4: Measurements of Dissolution Profiles of Sustained-Release Colchicine Formulations in Ethanol

To assess the potential for dose dumping, or dissolution of the composition in alcohol, the dissolution of the sustained release formulation of colchicine in ethanol was measured at various time points. The compositions were dissolved in 500 ml of three solutions, 5%, 20% and 40% ethanol, at 37 ° C and stirred continuously over a period of 6 hours. Samples were drawn at several time points to study the kinetics of the dissolution process of the drug substance within the hydrophilic matrix system. Colchicine content in the samples was analyzed using HPLC analysis.

### Example 5: Pharmacokinetic Properties of Sustained-Release Colchicine Formulations

The therapeutic effect of the sustained release formulation containing of colchicine is evaluated in a clinical study that is a multidose, randomized, cross-over study, which will evaluate bioavailability of about 3 different 0.50 mg sustained release formulations (e.g. Test Products 1,2,3 and FIG. 6) of colchicine to a 0.5 mg immediate release formulation (control product), administered to healthy volunteers. Test products 1, 2, and 3 used varying levels of retarding agent (i.e., Retalac), 23.3%, 26.6% or 30%, respectively, thereby having different sustained release profiles. The primary aim of the study is to assess pharmacokinetics (PK) of test and control drug in blood as well as in neutrophils or leucocytes.

Research objective: The primary objective of this study is to compare the pharmacokinetics of the test product vs. control product of colchicine in healthy human volunteers. In particular, levels of colchicine in blood (herein referred as to blood PK), neutrophils or leucocytes (herein referred as to neutrophil PK) will be assessed upon treatment with control and test product.

The hypothesis tested is that administration of an equal amount of colchicine in form of a sustained release tablet leads to lower peak levels (Cmax), while maintaining equal absolute bioavailability (area under the curve, AUC). Colchicine concentrations in neutrophils are measured as it is generally recognized that neutrophils which reconstitute 60-70% of leukocytes, play a central role in inflammatory responses in general and are thought to be major players in several diseases where colchicine is used as treatment. Therefore for the purpose of this experiment, either leucocytes or neutrophils may be analyzed. Hence, the term neutrophils as used herein also refers to, if used, leucocytes. As colchicine is known to preferentially accumulate in neutrophils and inhibit many of their pro-inflammatory functions, they are thought to be a major target of colchicine therapy. Therefore it is of special interest to know whether similar concentrations of colchicine are reached in isolated neutrophils or in leukocytes which would give information on potential bioequivalence. Therefore, blood will be drawn in various time points over the course of the study to check colchicine concentrations in blood and neutrophils.

General Study Design: This study is designed as a randomized, cross-over study. There will be 3 groups of patients (n=3× at least 8). Group 1 will receive control or test drug for about 8-14 days. After a wash out period, they will receive test drug 1 for about 8-14 days days. Group 2 will receive the control drug for the same time. After a wash out period, they will receive test drug 2 for the same time. Group 3 will receive the control drug for the same time. After a wash out period, they will receive test drug 3 for the same time. The study participants are treated on an in-patient basis for the first 24 h and on an out-patient basis for the remaining time. A sufficiently long wash-out period lies between the two trials.

Subject participation: There will be 3 groups of patients (n=3× at least 8). For each test drug, healthy volunteers will be randomized to one drug for the first round. After a sufficient wash out period, the same subjects will receive the control drug.

Study duration: The study drug is administered at a single dose/day for about 8-14 days consecutive days. The study consists of 24 h blood PK (high frequency data collection). Neutrophil PK is analysed in intervals that allow for conclusive determination of colchicine levels in the latter over the duration of the experiment.

Treatment regimen: Below is an example of what the treatment regime may look like.

| | Cycle 1 | Wash out | Cycle 2 |
|---|---|---|---|
| Group 1 | 14 days | 14 days | 14 days |
| | Control or test 1 | | Test 1 or control |
| Group 2 | 14 days | 14 days | 14 days |
| | Control or test 2 | | Test 2 or control |
| Group 3 | 14 days | 14 days | 14 days |
| | Control or test 3 | | Test 3 or control |

Research techniques and data analysis: Analytical chemistry techniques (HPLC) or immunological techniques (radioimmunoassay) may be utilized for the assessment of colchicine and optionally, its metabolites. Collected data is analyzed by adequate data management and statistics software.

Subject Population: The study population consists of male healthy volunteers (n=5-10/group). For inclusion in the study, the patient must be 25-40 years of age; not less than 60 kg and not more than 120 kg body weight; healthy; no major competing comorbidities or contraindication to colchicine therapy; willing to provide consent and be randomized into the study. Patients who meet the following criteria will be excluded: ongoing therapy with other anti-inflammatory/immunosuppressive drugs; treatment with drugs with known drug interactions with colchicine; renal/hepatic impairment; known hypersensitivity to colchicine.

Treatment schema: The drug is given once a day in the morning.

Data collection schema: For blood PK, blood will be collected over a period of 24 h at the beginning the study in short intervals (sufficient time points to establish pharmacokinetic data, e.g.: −1, +0.5 h, 1 h, 1.5 h, 2 h, 3 h, 4 h, 5 h, 7 h, 10 h, 12 h, 15 h and 24 h (before next application). Additional time points may be 24 h, and 10 days after drug withdrawal at the end of the study to examine the wash out phase of the drug. PK analysis includes colchicine blood concentrations. Adequate techniques for the isolation of blood and quantification of colchicine are applied.

For neutrophil PK, neutrophils will be isolated or purified from raw blood from several time points. Neutrophils will be collected at the beginning the study as well as at least once at the end of the study. PK analysis includes colchicine concentrations in isolated neutrophils.

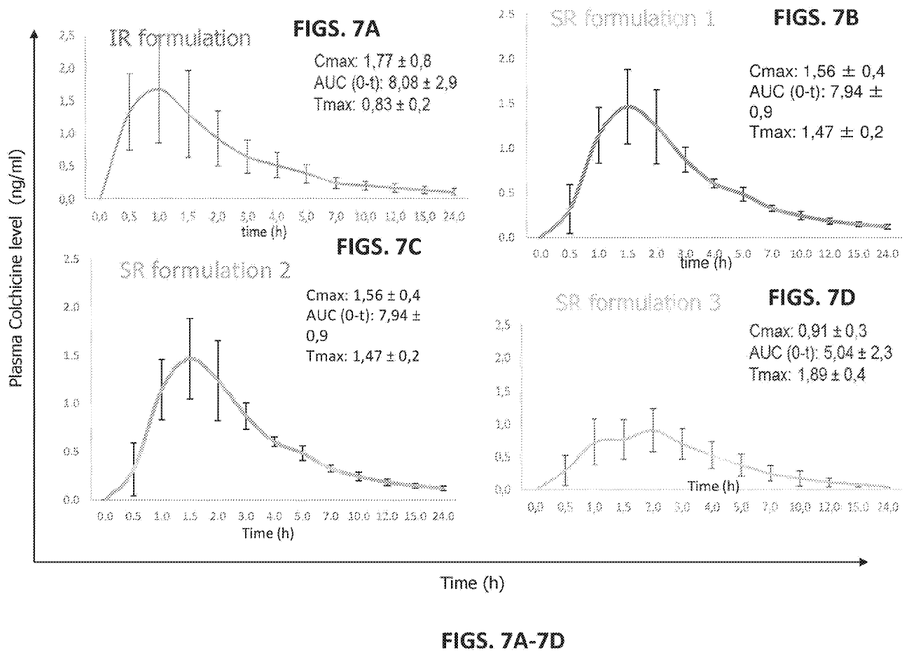

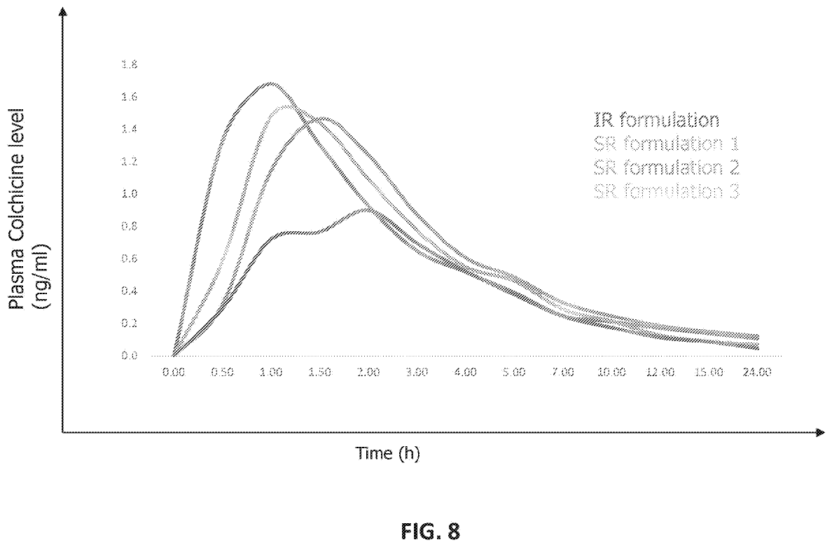

Results: The results are shown in FIGS. 7-9. FIGS. 7A-7D show plasma colchicine levels (ng/ml) as a function of time (hrs) in the immediate release formulation (FIG. 7A) as compared to the sustained release formulations (FIGS. 7B, 7C and 7D). The immediate release formulation of FIG. 7A has a Cmax of 1.77+/−0.8 ng/mL, an AUC (o-t) of 8.08+/−2.9 ng/ml and a Tmax of 0.83+/−0.2 hr. The sustained release formulation of FIG. 7B has a Cmax of 1.56+/−0.4 ng/ml, an AUC (o-t) of 7.94+/−0.9 ng/mL and a Tmax of 1.47+/−0.2 hr. The sustained release formulation of FIG. 7C has a Cmax of 1.56+/−0.4 ng/ml, an AUC (o-t) of 7.94+/−0.9 ng/ml and a Tmax of 1.47+/−0.2 hr. The sustained release formulation of FIG. 7D has a Cmax of 0.91+/−0.3 ng/ml, an AUC (o-t) of 5.04+/−2.3 ng/ml and a Tmax of 1.89+/−0.4 hr. FIG. 8 shows the overlapping profiles of the immediate release formulation and the three sustained release profiles. As can be seen, there is less inter-subject variation in plasma levels, lower Cmax and similar AUC for the sustained release formulation of FIG. 7B when compared to the immediate release formulation. Since the colchicine concentration on plasma increases and decreases more steadily and in a more controlled way, and the variability of colchicine levels in plasma from patient to patient is less, the safety and efficacy profile is more predicable. As a result, there will likely be fewer adverse side effects (e.g., gastrointestinal side effects) and/or reduced risk of toxicity (e.g., due to drug-drug interactions or co-morbidities such as renal and/or hepatic impairment).

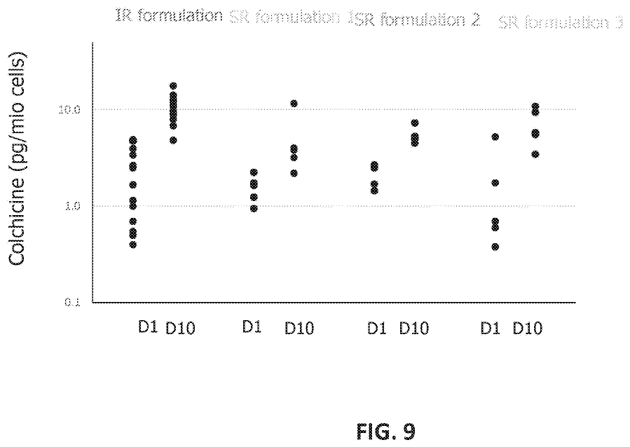

FIG. 9 shows the levels of colchicine in neutrophils on Day 1 and Day 10 in the immediate release formulation and the three sustained release formulations. As can be seen, the levels of colchicine increase from Day 1 to Day 10, suggesting that colchicine accumulates in neutrophils over time. The results further show that the levels in neutrophils are similar after 10 days regardless of the formulation, suggesting that the therapeutic effect will be equal for the tested sustained release and immediate release formulations (as the site of action of colchicine is neutrophils).

### Example 6: Therapeutic Effects of an Immediate Release Formulation in Patients with Cardiovascular Disease

To assess the therapeutic effects of an immediate release formulation in patients with cardiovascular disease, a prospective randomized observer blinded end-point trial was conducted to determine whether adding 0.5 mg/day of colchicine to standard secondary prevention therapies including aspirin and high dose statins reduces the risk of cardiovascular events in patients with objectively diagnosed and clinically stable coronary disease. This study is described in PCT/AU2013/001261 and is hereby incorporated in its entirety by reference.

Study Conduct and Design: The LoDoCo Trial was conducted under the auspices of the Heart Research Institute of Western Australia. It was designed by the principal investigators, registered with the Australian Clinical Trial Registry (12610000293066), and received ethics approval from the Human Research Ethics Committee at Sir Charles Gairdner Hospital Perth Western Australia in July 2008. There was no external funding source.

The study had a prospective randomized, open, blinded end-point design. Eligible consenting patients with established coronary disease presenting for routine clinical review were randomized to receive colchicine 0.5 mg/day or no colchicine without any other change to their medical therapy. All outcomes were evaluated by an experienced adjudicator blinded to the treatment allocation.

Study Size and Eligibility: It was planned to recruit a study population that would include 250 patients, 28 randomized to the control group and 250 patients randomized to treatment who were tolerant of colchicine for at least 4 weeks after the date of their randomization. Patients were eligible for inclusion if they met each of the following criteria: 1) angiographically proven coronary disease; 2) aged 35 to 85 years; 3) clinically stable for at least 6 months, 4) no major competing co-morbidities or contra-indication to colchicine therapy, 5) considered to be compliant with therapy and attending routine cardiology follow up appointments, and 6) willing to be consented and randomized into the study. Patients with a history of bypass surgery were only eligible if they had undergone bypass surgery more than 10 years before, or had angiographic evidence of graft failure or had undergone stenting since their bypass surgery. All patients signed informed consent before randomization.

Randomization: The randomization sequence was computer generated, kept concealed from the investigators at all times and was managed by a research assistant who had no involvement in the evaluation or management of study patients. Once the assistant received the consent form, the patients' demographic data were entered into the data base and the investigators and patients were advised in writing of the treatment group to which the patient had been assigned. Despite electing to use the lowest dose of colchicine available, it was anticipated that a number of patients would withdraw from therapy early after randomization due to gastrointestinal side effects. In order to ensure that the requisite number of patients in the treatment arm were actually tolerant of treatment, the protocol allowed for the research assistant to assign a newly recruited patient to treatment if a patient discontinued colchicine due to side effects in the first month. Patients who were intolerant to therapy remained in the study, and were followed in the usual manner and included in the primary intention to treat analysis.

Intervention: Patients randomized to active treatment were given a prescription for colchicine 0.5 mg daily by their referring cardiologist. The drug was dispensed by their usual chemist, and if requested, patients were reimbursed for the cost of these scripts. All other treatments were continued as usual.

Follow-up and definition of clinical outcomes: Patient compliance with treatment and outcome data were collected at routine follow up visits and at the time of any unplanned hospital admission. An acute coronary syndrome (ACS) was defined as either (a) Acute Myocardial Infarction (AMI), as evidenced by acute ischemic chest pain associated with a rise in serum troponin above the upper limit of normal or (b) Unstable Angina (UA), as evidenced by a recent acceleration of the patient's angina unassociated with a rise in serum troponin but associated with angiographic evidence of a change in the patient's coronary anatomy. (Unstable Angina Braunwald classification types IB and IIB). The ACS was characterized as being stent-related if there was evidence of significant in-stent stenosis or acute stent thrombosis. Out of Hospital Cardiac Arrest was defined as either a sudden death as evidenced on the patient's death certificate, or a non-fatal out of hospital cardiac arrest, defined as a recovery from sudden collapse associated with documented asystole, ventricular tachycardia or ventricular fibrillation. Noncardio-embolic ischemic stroke was defined as CT or MRI proven ischemic stroke adjudged by the treating neurologist as not being due to atrial fibrillation or intracranial hemorrhage.

The primary efficacy outcome was the composite, ACS, fatal or non-fatal out of hospital cardiac arrest or non-cardio-embolic ischemic stroke. Secondary outcomes were (a) individual components of the primary outcome, and (b) the components of ACS unrelated to stent disease.

Timelines: The pre-specified study duration was a minimum follow up of two years in all patients. Accordingly the study was closed on May 31, 2012. During May, all living patients were contacted by phone to collect compliance and outcome data from the last date of follow-up. Final outcome data were available in all patients and no patients were lost to follow up.

Statistical Power: Assuming that the control group had a combined event rate (ACS, out of hospital cardiac arrest or non cardio-embolic-ischemic stroke) of 8%, an accrual interval of 2 years and a follow-up after the accrual interval of 2 years, the planned sample size provided >80% power to detect a hazard ratio of <0.50 based on a two sided significance level of 5%.

Data analysis: Summary statistics, including mean and standard deviation were calculated for all baseline characteristics by treatment arm. All time to event outcomes were calculated in days by subtracting the date of randomization from either: (1) the date of event or death; or (2) the trial termination date for those patients not experiencing the defined event. As pre-specified, the primary efficacy analysis was based on the intention-to-treat principle. The intention-to-treat analysis included all randomized subjects and all events during the time from randomization to the trial termination. Trial termination date was fixed as May 31, 2012. A secondary pre-specified on-treatment analysis was also performed, based on patients who were both tolerant and compliant to therapy beyond the first month of randomization. All events during the time from randomization until non-compliance with colchicine treatment regimen were included in this analysis.

The time-to-first-event for all outcomes is presented using a Kaplan-Meier plot. The primary efficacy outcome was analyzed using a cox proportional hazards model including treatment group coded as control or colchicine. The secondary outcomes were analysed similarly. In addition, the primary analysis was stratified by gender, age, diagnosis of diabetes, past myocardial infarction, unstable angina, coronary bypass surgery, coronary angioplasty, and therapy with aspirin, clopidogrel or both, high dose statin therapy (defined as a dose of statin equivalent to atorvastatin of 40 mg or more), beta blockers, calcium blockers and ACE inhibitors.

Results: Between August 2008 and May 2010, 901 patients with stable coronary disease attending for routine out-patient cardiology review were assessed for eligibility for the study. Of these, 297 (33%) did not meet the entry criteria, 72 (8%) declined to participate and 532 (59%) were enrolled into the study, 250 of whom were randomized to the control group and 282 to treatment. Of those randomized to treatment 32 (11%) reported early intolerance, due to gastrointestinal side effects, and 7 patients subsequently reported that they chose not start therapy. All 532 randomized patients were followed for the duration of the study period which ranged from a minimum of 24 to a maximum of 44 months. Median follow up was 36 months.

Outcomes: A primary outcome occurred in 55/532 patients, including 15/282 (5.3%) patients assigned to colchicine treatment, and 40/250 (16%) patients assigned to the control group [hazard ratio 0.33, 95% CI; 0.18-0.59; p<0.001, number needed to treat 11). A sensitivity analysis was performed for the primary outcome, adjusting for the usage of calcium channel blockers and beta blocker therapy. These results were consistent with the primary analysis.

The effect of colchicine on the primary outcome was evident early and the benefits of colchicine continued to accrue throughout the follow up period. There was no evidence of differential treatment effects based on any of the clinical or therapeutic variables.

The reduction in the primary outcome was largely driven by the reduction in the number of patients presenting with an ACS, (13/282 (4.6%) vs. 34/250 (13.4%), hazard ratio 0.33; 95% CI; 0.18-0.63; p<0.001). Out of hospital cardiac arrest and non-cardio-embolic ischemic stroke were infrequent but were also reduced in the treatment group.

Of the 47 patients who presented with an ACS, the event was stent related in 8 (17%) (2 in each group had evidence of acute stent thrombosis and 2 in each group had evidence of significant in-stent stenosis). Further analysis confirmed that patients randomized to treatment were less likely to present with an ACS unrelated to stent disease (9/282 (3.2%) vs. 30/250 (12%) hazard ratio 0.26, 95% CI; 0.12-0.55; p<0.001), be it associated with an AMI (4/282 (1.4%) vs. 14/250 (5.6%) hazard ratio 0.25, 95% CI; 0.08-0.76; p=0.014) or UA (5/282 (1.8%) vs. 16/250 (6.4%) hazard ratio 0.27, 95% CI; 0.10-0.75; p=0.011).

Of 39 patients randomized to treatment who did not receive therapy beyond the first month due to early intolerance or non-compliance, 4 (10%) presented with an ACS due to acute stent thrombosis (n=1) and UA (n=3). Patients who were both compliant and tolerant to therapy beyond the first month of randomization had significantly fewer events than the control patients (11/243 (4.5%) vs. 40/250 (16%) hazard ratio 0.29, 95% CI; 0.15-0.56; p<0.001). The results of all on-treatment analyses were consistent with those based upon the intention to treat analyses.

Ten patients in the control group died compared with 4 patients in the colchicine group. Of the 10 controls, 5 died of presumed cardiac cause; 2 following an out-of-hospital cardiac arrest, 2 from cardiogenic shock following myocardial infarction, and 1 following bypass surgery. All 4 patients in the colchicine group died of non-cardiac causes.

This trial demonstrates that the addition of colchicine 0.5 mg/day to standard therapy in patients with stable coronary disease significantly reduces the risk of a cardiovascular event, including an ACS, out of hospital cardiac arrest and non-cardio-embolic ischemic stroke. The benefits of colchicine were achieved on a background of widespread use of effective secondary prevention strategies, including high dose statins, as evidenced by the low event rate in the control group. The effect of adding colchicine became evident early, continued to accrue over time and was largely driven by a reduction in ACS unrelated to stent disease.

### Example 7: Dose Adjustments of Sustained Release Colchicine Formulations

To determine the proper dose amounts, the sustained released formulation may be administered to patient populations with different body weights. Assuming that a 0.5 mg tablet gives a certain plasma level of colchicine in average weight patients and that this particular level should be reached in every patient to achieve efficacy, one can perform PK analysis of a 0.5 mg tablet in different weight groups. It is expected that levels in blood (Cmax and AUC) in heavy patients are lower, which indicates that dose adjustments towards higher doses is necessary.

Research objective: The primary objective of this study is to compare the pharmacokinetics of a 0.5 mg colchicine tablet in healthy human volunteers of at least 2 different body weight groups. In particular, levels of colchicine in blood will be assessed upon treatment. The hypothesis tested is that administration of an equal amount of colchicine in leads to different colchicine levels depending to body weight. Body weight and colchicine levels are inverse correlated.

General Study Design: This study is designed as a randomized, cross-over study. n=at least 8 subjects are selected according to body weight. If 2 groups are made, then 50% will be of low weight (e.g. below 65 kg) and 50% of high weight (e.g. above 90 kg).

Study duration: The study drug is administered once. The study consists of 24 h blood PK (high frequency data collection).

Research techniques and data analysis: Analytical chemistry techniques (HPLC) or immunological techniques (radioimmunoassay) may be utilized for the assessment of colchicine. Collected data is analyzed by adequate data management and statistics software.

Subject Population: The study population consists of male healthy volunteers. For inclusion in the study, the patient must be 25-40 years of age; not less than 50 kg and not more than 120 kg body weight; healthy; no major competing comorbidities or contraindication to colchicine therapy; willing to provide consent and be randomized into the study. Patients who meet the following criteria will be excluded: ongoing therapy with other anti-inflammatory/immunosuppressive drugs; treatment with drugs with known drug interactions with colchicine; renal/hepatic impairment; known hypersensitivity to colchicine.

Treatment schema: The drug is given once in the morning of the study.

Data collection schema: For blood PK, blood will be collected over a period of 24 h at the beginning the study in short intervals (sufficient time points to establish pharmacokinetic data, e.g.: −1, +0.5 h, 1 h, 1.5 h, 2 h, 3 h, 4 h, 5 h, 7 h, 10 h, 12 h, 15 h and 24 h (before next application). Additional time points may be 24 h, and 72 h after drug withdrawal at the end of the study to examine the wash out phase of the drug.

### Example 8: Therapeutic Effects of Colchicine in Patients with Post-COVID-19 Vaccination Inflammatory Syndrome

To evaluate the efficacy of colchicine in treating persistent systemic inflammation following COVID-19 vaccination, a randomized, double-blind, placebo-controlled clinical study is proposed. The objective of this hypothetical study is to determine whether once-daily administration of 0.5 mg colchicine can reduce systemic inflammatory biomarkers and improve clinical symptoms in patients experiencing post-vaccination inflammatory syndromes.

Study Design: The proposed study would enroll approximately 150 adult participants who report persistent inflammatory symptoms (e.g., fatigue, myalgia, arthralgia, chest discomfort) arising within 12 weeks after receiving a COVID-19 vaccine. Subjects must have elevated levels of one or more inflammatory biomarkers (e.g., hs-CRP, IL-6, TNF-α). Participants will be randomized 1:1 to receive either 0.5 mg/day colchicine or a matched placebo for a 6-month treatment period. Biomarker assays and clinical symptom scoring will be conducted at baseline, 3 months, and 6 months.

Eligibility Criteria: Inclusion criteria include adults aged 18-65 years who received at least one dose of a COVID-19 vaccine and subsequently developed persistent inflammatory symptoms lasting more than 6 weeks. Laboratory confirmation of systemic inflammation (e.g., hs-CRP>2 mg/L or IL-6>3 μg/mL) is required. Exclusion criteria include prior intolerance to colchicine, known autoimmune disease, use of systemic immunosuppressive therapy, or clinically significant renal or hepatic impairment.

Primary and Secondary Endpoints: The primary endpoint will be the percent reduction in hs-CRP levels from baseline to 6 months.

Secondary endpoints will include:
Reduction in IL-6 and TNF-α levels
Improvement in symptom severity scores using a validated patient-reported outcome measure
Improvement in fatigue severity scale and 6-minute walk test
Incidence of adverse events and treatment discontinuation rates

Statistical Considerations: The study is powered to detect a 25% reduction in mean hs-CRP levels in the treatment group compared to placebo, assuming a standard deviation of 40%, with 75 participants per arm providing >80% power at a two-sided alpha of 0.05. Analyses will be performed on an intention-to-treat basis. Exploratory subgroup analyses will assess biomarker response in participants with myocarditis or pericarditis.

Expected Outcomes: It is anticipated that patients receiving colchicine will demonstrate significant reductions in systemic inflammation markers compared to placebo. Improvements in functional capacity and quality-of-life scores are also expected. Safety and tolerability will be closely monitored, particularly gastrointestinal side effects.

Conclusion: This hypothetical study is designed to support the role of colchicine as a therapeutic option for managing persistent systemic inflammation following COVID-19 vaccination. By targeting key inflammatory pathways, colchicine may provide symptom relief and reduce immune-mediated complications in affected individuals.

Many modifications and other embodiments set forth herein will come to mind to one skilled in the art to which these embodiments pertain having the benefit of the teachings presented in the foregoing descriptions and the associated drawings. Therefore, it is to be understood that the described methods and formulations are not to be limited to the specific embodiments disclosed and that modifications and other embodiments are intended to be included within the scope of the appended claims and list of embodiments disclosed herein. Although specific terms are employed herein, they are used in a generic and descriptive sense only and not for purposes of limitation.

What is claimed is:

1. A method of treating chronic inflammation in a human subject, which comprises:
administering to the subject a daily oral dose of about 0.5 mg of colchicine, or a pharmaceutically acceptable salt thereof, or a combination thereof,
wherein levels of one or more of inflammation biomarkers high-sensitivity C-reactive protein (hs-CRP), interleukin-1β (IL-1β), interleukin-6 (IL-6), tumor necrosis factor-alpha (TNF-α), and myeloperoxidase (MPO) are present in the subject at levels detectably higher than basal level.

2. The method of claim 1, wherein the level of hs-CRP in the subject is above 2 mg/L.

3. The method of claim 1, wherein the IL-6 level in the subject is above 3 μg/mL.

4. The method of claim 1, wherein the subject possesses clinical symptoms including one or more of fatigue, joint pain, myalgia, cognitive impairment, and low-grade fever.

5. The method of claim 1, wherein administering is for at least 3 months.

6. The method of claim 5, wherein administering is for at least 12 months.

7. The method of claim 1, further comprising administering one or more of non-steroidal anti-inflammatory drugs (NSAIDs), corticosteroids, and interleukin-6 inhibitors.

8. The method of claim 1, further comprising determining protein levels of the inflammation biomarkers during and/or after treatment on a periodic basis, wherein determining comprises assaying blood of the subject with blood-based assays, and wherein periodically comprises every 4 to 12 weeks during and/or after treatment.

9. The method of claim 1, wherein the hs-CRP levels in the subject decrease by at least 30% after 8 weeks.

10. The method of claim 1, wherein the colchicine is in a form of an immediate-release tablet, an extended-release tablet, or an oral suspension.

11. The method of claim 1, wherein the subject has, prior to the method, the protein levels of the inflammation biomarkers of the subject failed to decrease when the subject was administered NSAIDs and/or corticosteroids.

12. The method of claim 1, wherein the subject suffers from inflammation flares, and wherein the method reduces the flares by frequency or severity, and wherein the subject suffers from rheumatoid arthritis, systemic lupus erythematosus, and/or inflammatory bowel disease.

13. A method of treating chronic inflammation in a human subject following COVID-19 vaccination, the method comprising:
administering to the subject a daily oral dose of about 0.5 mg of colchicine, a pharmaceutically acceptable salt thereof, or a combination thereof,
wherein the subject exhibits persistent systemic inflammation that arises within 12 weeks of receiving a COVID-19 vaccine,
wherein protein levels of one or more of inflammation biomarkers high-sensitivity C-reactive protein (hs-CRP), interleukin-1β (IL-1β), interleukin-6 (IL-6), tumor necrosis factor-alpha (TNF-α), and myeloperoxidase (MPO) are present in the subject at levels detectably higher than basal level, and
wherein the protein levels of the inflammatory biomarkers are reduced after the method.

14. The method of claim 13, wherein the COVID-19 vaccine is an mRNA-based vaccine.

15. The method of claim 13, wherein the COVID-19 vaccine is BNT162b2, mRNA-1273, or a vaccine comprising lipid nanoparticle-encapsulated mRNA.

16. The method of claim 13, wherein the subject developed myocarditis, pericarditis, or myopericarditis after vaccination.

17. The method of claim 13, wherein the administering is initiated within 3 months of the onset of the persistent systemic inflammation.

18. The method of claim 13, wherein the subject exhibits one or more of the following clinical symptoms of chronic inflammation: fatigue, arthralgia, myalgia, chest discomfort, shortness of breath, and cognitive impairment.

19. The method of claim 13, wherein the administering occurs for at least 3 months.

20. The method of claim 13, wherein the hs-CRP is reduced by at least 30% after the method is conducted for 8 weeks.

21. The method of claim 13, wherein prior to the method, the protein levels of the inflammation biomarkers failed to decrease when the subject was administered corticosteroids and/or NSAIDs.

22. The method of claim 13, wherein colchicine is administered in combination with one or more of corticosteroids, IL-1 inhibitors, and IL-6 inhibitors.

23. The method of claim 13, wherein the persistent systemic inflammation in the subject is diagnosed by imaging of affected organs or tissues, and wherein imaging comprises imaging of cardiac tissue by magnetic resonance imaging (MRI) for myocarditis or positron emission tomography-computed tomography (PET-CT).

24. The method of claim 13, further comprising determining protein levels of the inflammation biomarkers during and/or after treatment, wherein determining comprises assaying blood of the subject with blood-based assays.

25. A method of treating chronic inflammation or Long COVID in a human subject following COVID-19 infection, the method comprising:
administering to the subject a daily oral dose of about 0.5 mg of colchicine, a pharmaceutically acceptable salt thereof, or a combination thereof,
wherein the subject exhibits clinical symptoms of Long COVID that arises within 3 months of COVID-19 infection,
wherein levels of one or more of inflammation biomarkers C-reactive protein (CRP), interleukin-1 (IL-1), interleukin-6 (IL-6), and tumor necrosis factor-alpha (TNF-α), are present in the subject at levels detectably higher than basal level, and
wherein the levels of the inflammatory biomarkers are reduced after the method.

26. The method of claim 25, wherein the subject developed myocarditis, pericarditis, or myopericarditis after COVID infection.

27. The method of claim 25, wherein the administering is initiated within 3 months of the onset of the clinical symptoms of Long COVID.

28. The method of claim 25, wherein the clinical symptoms of Long COVID are one or more of fatigue, brain fog, forgetfulness, chest pain, post-exertion malaise (PEM), and shortness of breath or difficulty breathing.

29. The method of claim 25, wherein the administering occurs once per day for at least 3 months.

30. The method of claim 25, wherein the CRP is reduced by at least 30% after the method is conducted for at least 8 weeks.

31. The method of claim 25, wherein prior to the method, the levels of the inflammation biomarkers failed to decrease when the subject was administered corticosteroids and/or NSAIDs after diagnosis of Long COVID.

32. The method of claim 25, wherein colchicine is administered in combination with one or more of corticosteroids, IL-1 inhibitors, and IL-6 inhibitors.

33. The method of claim 25, further comprising determining protein levels of the inflammation biomarkers during and/or after the method, wherein determining comprises assaying blood of the subject for the protein level of the inflammation biomarkers.
